(19) United States
(12) Patent Application Publication
(10) Pub. No.: US 2006/0161185 A1
(43) Pub. Date: Jul. 20, 2006

(54) Methods and apparatus for transmitting force to an end effector over an elongate member

(75) Inventors: Vahid Saadat, Saratoga, CA (US); Richard C. Ewers, Fullerton, CA (US); Ruey-Feng Peh, Singapore (SG); Tracy D. Maahs, Rancho Santa Margarita, CA (US)

Correspondence Address: LEVINE BAGADE LLP, 2483 EAST BAYSHORE ROAD, SUITE 100, PALO ALTO, CA, 94303, US

(73) Assignee: USGI Medical Inc., San Clemente, CA (US)

(21) Appl. No.: 11/035,993

(22) Filed: Jan. 14, 2005

(51) Int. Cl. A61B 17/08

(52) U.S. Cl. 606/153

(57) ABSTRACT

Apparatus and methods for conveying or transmitting force or energy to a medical end effector coupled to a flexible or rigid shaft are described herein. One variation of such apparatus may be used to manipulate tissue and create a tissue fold and may generally comprise an elongate tubular member having an end effector disposed thereon. The end effector may comprise a tissue engagement member adapted to engage tissue, a first stabilizing member and a second stabilizing member positioned at the tubular member distal end, and a launch tube adapted to pivot about the first stabilizing member. Elements of the end effector may be actuable via various force transmission elements and/or mechanisms. Such force transmission elements preferably are integrated into and/or are actuable via a handle. The force transmission mechanisms may be utilized to actuate and/or transmit force to alternative medical end effectors coupled to flexible or rigid shafts.

### BACKGROUND OF THE INVENTION

Field of the Invention

The present invention relates to methods and apparatus for conveying or transmitting force to a medical end effector over a flexible or rigid member. The methods and apparatus may, for example, be used to form and secure gastrointestinal (“GI”) tissue folds, e.g., to reduce the effective cross-sectional area of a gastrointestinal lumen or otherwise treat a region of gastrointestinal tissue.

Morbid obesity is a serious medical condition pervasive in the United States and other countries. Its complications include hypertension, diabetes, coronary artery disease, stroke, congestive heart failure, multiple orthopedic problems and pulmonary insufficiency with markedly decreased life expectancy.

A number of surgical techniques have been developed to treat morbid obesity, e.g., bypassing an absorptive surface of the small intestine, or reducing the stomach size. However, many conventional surgical procedures may present numerous life-threatening post-operative complications, and may cause atypical diarrhea, electrolytic imbalance, unpredictable weight loss and reflux of nutritious chyme proximal to the site of the anastomosis.

Furthermore, the sutures or staples that are often used in these surgical procedures typically require extensive training by the clinician to achieve competent use, and may concentrate significant force over a small surface area of the tissue, thereby potentially causing the suture or staple to tear through the tissue. Many of the surgical procedures require regions of tissue within the body to be approximated towards one another and reliably secured. The gastrointestinal lumen includes four tissue layers, wherein the mucosa layer is the inner-most tissue layer followed by connective tissue, the muscularis layer and the serosa layer.

One problem with conventional gastrointestinal reduction systems is that the anchors (or staples) should engage at least the muscularis tissue layer in order to provide a proper foundation. In other words, the mucosa and connective tissue layers typically are not strong enough to sustain the tensile loads imposed by normal movement of the stomach wall during ingestion and processing of food. In particular, these layers tend to stretch elastically rather than firmly hold the anchors (or staples) in position, and accordingly, the more rigid muscularis and/or serosa layer should ideally be engaged. This problem of capturing the muscularis or serosa layers becomes particularly acute where it is desired to place an anchor or other apparatus transesophageally rather than intra-operatively, since care must be taken in piercing the tough stomach wall not to inadvertently puncture adjacent tissue or organs.

One conventional method for securing anchors within a body lumen to the tissue is to utilize sewing devices to suture the stomach wall into folds. This procedure typically involves advancing a sewing instrument through the working channel of an endoscope and into the stomach and against the stomach wall tissue. The contacted tissue is then typically drawn into the sewing instrument where one or more sutures or tags are implanted to hold the suctioned tissue in a folded condition typically known as a plication. Another method involves manually creating sutures for securing the plication.

One of the problems associated with these types of procedures is the time and number of intubations needed to perform the various procedures endoscopically. Another problem is the time required to complete a plication from the surrounding tissue with the body lumen. In the period of time that a patient is anesthetized, procedures such as for the treatment of morbid obesity or for GERD must be performed to completion. Accordingly, the placement and securement of the tissue plication should ideally be relatively quick and performed with a maximum level of confidence.

Another problem with conventional methods involves ensuring that the staple, knotted suture, or clip is secured tightly against the tissue and that the newly created plication will not relax under any slack which may be created by slipping staples, knots, or clips. Other conventional tissue securement devices such as suture anchors, twist ties, crimps, etc. are also often used to prevent sutures from slipping through tissue.

Many of these types of devices are typically large and unsuitable for low-profile delivery through the body, e.g., transesophageally. This may be due to difficulties in applying, deploying and/or deforming such devices with low-profile end effectors disposed at significant distances from a medical practitioner, i.e., due to an inability to convey adequate force to the devices and/or end effectors along desired vectors across the significant distances. These difficulties may be exacerbated when the end effectors are coupled to the distal ends of flexible shafts. It is expected that enhanced capabilities for transmitting or conveying force to a medical device end effector coupled to a flexible or rigid shaft would facilitate myriad minimally invasive procedures, such as endoluminal treatment for morbid obesity.

### BRIEF SUMMARY OF THE INVENTION

In creating tissue plications, a tissue plication tool having a distal tip may be advanced (transorally, transgastrically, etc.) into the stomach. The tissue may be engaged or grasped, and the engaged tissue may be moved to a proximal position relative to the tip of the device, thereby providing a substantially uniform plication of predetermined size. In order to first create the plication within a body lumen of a patient, various methods and devices may be implemented. The anchoring and securement devices may be delivered and positioned via an endoscopic or laparoscopic endoluminal apparatus that engages a tissue wall of the gastrointestinal lumen, creates one or more tissue folds, and disposes one or more of the anchors through the tissue fold(s). The tissue anchor(s) may be disposed through the muscularis and/or serosa layers of the gastrointestinal lumen.

One variation of an apparatus that may be used to manipulate tissue and create a tissue fold may generally comprise an elongate tubular member having a proximal end, a distal end, and a length therebetween; and an end effector. The end effector may comprise a tissue engagement member in one variation, which is slidably disposed through the tubular member, having a distal end adapted to engage tissue, an upper or first stabilizing member and a lower or second stabilizing member positioned at the tubular member distal end and adapted to stabilize tissue therebetween, and a launch tube adapted to pivot about the first stabilizing member. The first and second stabilizing members preferably are adapted to be angled relative to a longitudinal axis of the elongate tubular member.

The end effector may be manipulated and articulated through various mechanisms. One such assembly that integrates each of the functions into a singular unit may comprise a handle assembly, which is connected via the tubular member to elements of the end effector. Such a handle assembly optionally may be configured to separate from the tubular member, thus allowing for reusability of the handle. An articulation control may also be positioned on the handle to provide for selective articulation of the extension members and/or other elements of the end effector.

One particular variation of the handle assembly may have a handle enclosure formed in a tapered configuration, which is generally symmetrically-shaped about a longitudinal axis extending from the distal end to the proximal end of the handle assembly. The symmetric feature may allow for the handle to be easily manipulated by the user regardless of the orientation of the handle enclosure during a tissue manipulation procedure.

To articulate the multiple features desirably integrated into a singular handle assembly, e.g., advancement and/or deployment of the launch tube, anchor assembly, needle assembly, articulation of the extension members and end effector, etc., a specially configured locking mechanism may be located within the handle enclosure. Such a locking mechanism may generally be comprised of an outer sleeve disposed about inner sleeve where the outer sleeve has a diameter, which allows for its unhindered rotational and longitudinal movement relative to the inner sleeve. A needle deployment locking control may extend radially from the outer sleeve and protrude externally from the enclosure for manipulation by the user. The outer sleeve may also define a needle assembly travel path along its length. The travel path may define the path through which the needle assembly may traverse in order to be deployed.

The needle assembly may define one or more guides protruding from the surface of the assembly, which may be configured to traverse within the travel path. The inner sleeve may also define guides protruding from the surface of the inner sleeve for traversal within grooves defined in the handle enclosure. Moreover, the outer sleeve is preferably disposed rotatably about the inner sleeve such that the outer sleeve and inner sleeve are configured to selectively interlock with one another in a corresponding manner when the locking control is manipulated into specified positions.

Elements of the end effector may be actuable via various force transmission elements described hereinafter. Such force transmission elements optionally may be integrated into and/or actuable via the handle. It should be understood that the force transmission elements optionally may be utilized to actuate and/or convey force to alternative medical end effectors coupled to flexible or rigid shafts.

### BRIEF DESCRIPTION OF THE DRAWINGS

FIG. 1A shows a side view of one variation of a tissue plication apparatus which may be used to create tissue plications and to deliver cinching or locking anchors into the tissue.

FIGS. 1B and 1C show detail side and perspective views, respectively, of the tissue approximation assembly of the device of FIG. 1A.

FIGS. 2A to 2D show side views, partially in section, of the tissue plication apparatus of FIG. 1 creating a tissue plication.

FIG. 3A shows a cross-sectional side view of an anchor delivery assembly delivering a basket-type anchor into or through a tissue fold.

FIG. 3B shows a cross-sectional side view of multiple tissue folds which may be approximated towards one another and basket anchors as being deliverable through one or both tissue folds.

FIGS. 4A and 4B show side views of one variation of the tissue manipulation assembly having cam-actuated extension members.

FIGS. 4C and 4D show detail views of the cam-actuation for the assembly of FIGS. 4A and 4B.

FIGS. 5A and 5B show side views of another variation of extension members which are biased towards one another.

FIGS. 6A and 6B show side views of another variation of extension members which are actuated via a linkage assembly.

FIGS. 7A to 7C show side views of another variation of extension members which are actuatable via one or more hinged arms interconnecting the extension members.

FIGS. 8A and 8B show side views of another variation where one or more extension members are biased away from one another.

FIGS. 9A and 9B show side views of another variation where one or more extension members are configured to be passively biased.

FIGS. 10A and 10B show side views of another variation of extension members which are actuatable via a translatable sleeve.

FIG. 11 shows a side view of a tissue manipulation assembly with a lower extension member having a longer length than the upper extension member.

FIG. 12 shows a side view of another variation where one or both extension members may have tips atraumatic to tissue.

FIGS. 13A and 13B views of a variation of lower extension members which may be configured to be actuatable.

FIG. 13C show a top view of a lower extension member which may be configured into a “C” shape.

FIGS. 14A and 14B show perspective and top views of a lower extension member having one or more energize-able wires disposed thereon for tissue ablation.

FIGS. 15A to 15E show side views, partially in section, of the apparatus of FIG. 14 creating and securing a tissue plication, while initiating a wound healing response.

FIGS. 16A to 16C show side views of a tissue manipulation assembly which may be configured to articulate into an angle relative to the tubular body.

FIGS. 17A to 17C show partial side views of variations of a handle for controlling and articulating the tissue manipulation assembly.

FIGS. 18A to 18C show top, side, and cross-sectional views, respectively, of another variation of a handle having a multi-position locking and needle assembly advancement system.

FIG. 18D shows an assembly view of the handle of FIG. 18A connected to the tissue manipulation assembly via a rigid or flexible tubular body or shaft.

FIGS. 19A and 19B show perspective and cross-sectional views, respectively, of another variation of a handle having a reversible configuration.

FIGS. 20A and 20B show partial cross-sectional side and detail views, respectively, of another variation of a handle having a pivotable articulation control.

FIG. 21A shows a side view of the handle of FIG. 20A having the multi-position locking and needle assembly advancement system.

FIGS. 21B to 21D show end views of the handle of FIG. 21A and the various positions of the multi-position locking and needle assembly advancement system.

FIG. 22A shows a perspective view of one variation of the multi-position locking and needle assembly advancement system.

FIGS. 22B to 22E show illustrative side views of the system of FIG. 22A configured in various locking and advancement positions.

FIG. 23 illustrates a side view of a needle deployment assembly which may be loaded or advanced into an approximation assembly.

FIG. 24A shows a side view of one variation of a needle deployment assembly.

FIG. 24B shows an exploded assembly of FIG. 24A in which the tubular sheath is removed to reveal the anchor assembly and elongate pusher element.

FIGS. 25A and 25B show partial cross-sectional side views of a shuttle element advanced within the needle assembly housing.

FIGS. 26A and 26B illustrate one variation of deploying the anchors using the needle assembly.

FIG. 26C illustrates a partial cross-sectional view of one variation of the needle and anchor assemblies positioned within the launch tube.

FIG. 27 is a schematic view of apparatus comprising a medical end effector coupled to a handle via an elongate tubular body.

FIG. 28 is a side view, partially in section, of a transmission element or mechanism for transmitting force or energy to a medical end effector.

FIGS. 29A and 29B are side views, partially in section, of a transmission mechanism that transmits and converts rotational motion into translation motion via a lead screw.

FIGS. 30A and 30B are side views, partially in section, of a transmission mechanism that converts rotational motion into translational motion and actuates a linkage to initiate a more complex motion that actuates a tissue grasper.

FIGS. 31A and 31B are side views, partially in section, of an alternative embodiment of the apparatus of FIG. 30 comprising a tissue manipulation assembly having extension members.

FIGS. 32A and 32B are side views, partially in section, of a transmission mechanism that facilitates coordinated reorientation or pivoting of extension members of a tissue manipulation assembly.

FIGS. 33A and 33B are side views, partially in section, of a transmission mechanism that converts hydraulic energy into mechanical energy.

FIGS. 34A and 34B are side views, partially in section, of another embodiment of a hydraulically-actuated medical end effector.

FIGS. 35A and 35B are, respectively, a side-sectional view and a cross-sectional view, of another hydraulically-actuated end effector.

FIGS. 36A and 36B are side views, partially in section, of yet another hydraulically-actuated end effector.

FIGS. 37A and 37B are side views, partially in section, of a transmission mechanism that converts electrical energy into rotational and translational mechanical energy.

FIGS. 38A and 38B are side views, partially in section, of a transmission mechanism that converts electrical energy into a complex mechanical motion.

FIGS. 39A and 39B are side views, partially in section, of a motor-actuated linkage.

FIGS. 40A and 40B are side views, partially in section, of a transmission mechanism comprising a column of ball-bearings.

FIGS. 41A and 41B are, respectively, a side-sectional view and a side-sectional detail view, of a crimping or grasping end effector actuated via a ball-bearing column transmission mechanism.

FIGS. 42A and 42B are side views, partially in section, of a transmission mechanism utilizing geometric constraints.

FIGS. 43A-43D are side views, partially in section, illustrating apparatus and a method for deforming a crimp with a linkage assembly actuated via a lead screw transmission mechanism.

FIGS. 44A and 44B are side views, partially in section, of an alternative embodiment of the apparatus and method of FIG. 43.

FIGS. 45A and 45B are side views, partially in section, of a linkage actuated via translational motion.

FIG. 46 is a schematic view of a generic transmission mechanism for transmitting force or energy to a medical end effector.

### DETAILED DESCRIPTION OF THE INVENTION

In creating tissue plications, a tissue plication tool having a distal tip may be advanced (transorally, transgastrically, etc.) into the stomach. The tissue may be engaged or grasped and the engaged tissue may be moved to a proximal position relative to the tip of the device, thereby providing a substantially uniform plication of predetermined size. Examples of creating and forming tissue plications may be seen in further detail in U.S. patent application Ser. No. 10/735,030 filed Dec. 12, 2003, which is incorporated herein by reference in its entirety.

In order to first create the plication within a body lumen of a patient, various methods and devices may be implemented. The anchoring and securement devices may be delivered and positioned via an endoscopic apparatus that engages a tissue wall of the gastrointestinal lumen, creates one or more tissue folds, and disposes one or more of the anchors through the tissue fold(s). The tissue anchor(s) may be disposed through the muscularis and/or serosa layers of the gastrointestinal lumen.

Generally, in creating a plication through which a tissue anchor may be disposed within or through, a distal tip of a tissue plication apparatus may engage or grasp the tissue and move the engaged tissue to a proximal position relative to the tip of the device, thereby providing a substantially uniform plication of predetermined size.

Formation of a tissue fold may be accomplished using at least two tissue contact areas that are separated by a linear or curvilinear distance, wherein the separation distance between the tissue contact points affects the length and/or depth of the fold. In operation, a tissue grabbing assembly end effector engages or grasps the tissue wall in its normal state (i.e., non-folded and substantially flat), thus providing a first tissue contact area. The first tissue contact area then is moved to a position proximal of a second tissue contact area to form the tissue fold. A tissue anchor assembly then may be extended across the tissue fold at the second tissue contact area. Optionally, a third tissue contact point may be established such that, upon formation of the tissue fold, the second and third tissue contact areas are disposed on opposing sides of the tissue fold, thereby providing backside stabilization during extension of the anchor assembly across the tissue fold from the second tissue contact area.

The first tissue contact area may be utilized to engage and then stretch or rotate the tissue wall over the second tissue contact area to form the tissue fold. The tissue fold then may be articulated to a position where a portion of the tissue fold overlies the second tissue contact area at an orientation that is substantially normal to the tissue fold. A tissue anchor then may be delivered across the tissue fold at or near the second tissue contact area. An apparatus which is particularly suited to deliver the anchoring and securement devices described herein may be seen in further detail in co-pending U.S. patent application Ser. No. 10/840,950 filed May 7, 2004, which is incorporated herein by reference in its entirety.

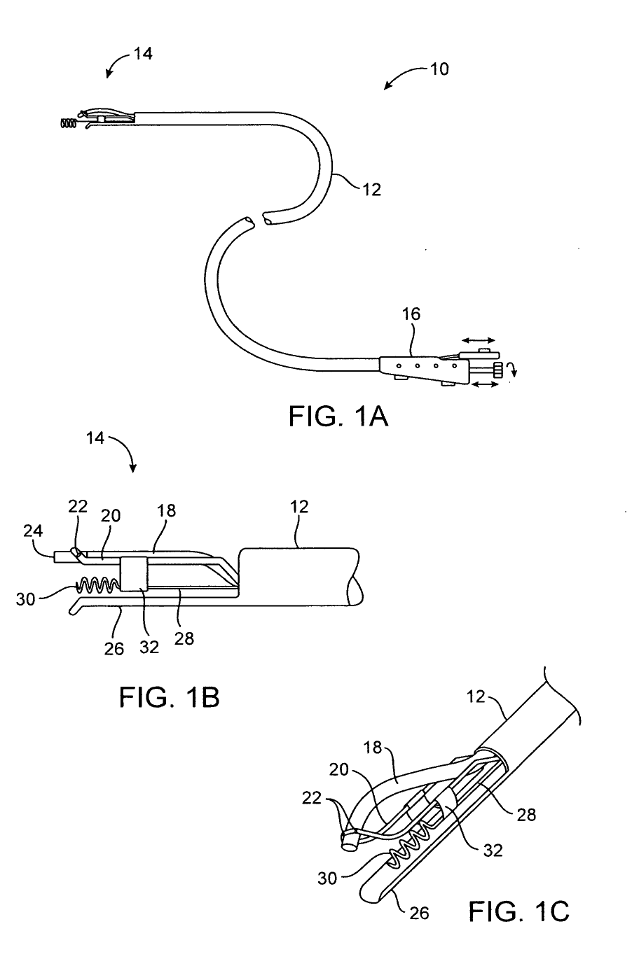

An illustrative side view of a tissue plication assembly 10 which may be utilized with the tissue anchors described herein is shown in FIG. 1A. The plication assembly 10 generally comprises a catheter or tubular body 12 which may be configured to be sufficiently flexible for advancement into a body lumen, e.g., transorally, percutaneously, laparoscopically, etc. Tubular body 12 may be configured to be torqueable through various methods, e.g., utilizing a braided tubular construction, such that when handle 16 is manipulated and rotated by a practitioner from outside the body, the torquing force is transmitted along body 12 such that the distal end of body 12 is rotated in a corresponding manner.

Tissue manipulation assembly or end effector 14 is located at the distal end of tubular body 12 and is generally used to contact and form the tissue plication, as mentioned above. FIG. 1B shows an illustrative detail side view and FIG. 1C shows a perspective view of tissue manipulation assembly/end effector 14 which shows launch tube 18 extending from the distal end of body 12 and in-between the arms of upper extension member or bail 20. Launch tube 18 may define launch tube opening 24 and may be pivotally connected near or at its distal end via hinge or pivot 22 to the distal end of upper bail 20. Lower extension member or bail 26 may similarly extend from the distal end of body 12 in a longitudinal direction substantially parallel to upper bail 20. Upper bail 20 and lower bail 26 need not be completely parallel so long as an open space between upper bail 20 and lower bail 26 is sufficiently large enough to accommodate the drawing of several layers of tissue between the two members.

Several variations of the tissue plication assembly 10 and some of the various apparatus used therewith are disclosed in further detail herein below as well as in U.S. patent application Ser. No. 10/954,666 filed Sep. 29, 2004, which is incorporated herein by reference in its entirety.

Upper bail 20 is shown in the figure as an open looped member and lower bail 26 is shown as a solid member; however, this is intended to be merely illustrative and either or both members may be configured as looped or solid members. Tissue acquisition member 28 may be an elongate member, e.g., a wire, hypotube, etc., which terminates at a tissue grasper or engager 30, in this example a helically-shaped member, configured to be reversibly rotatable for advancement into the tissue for the purpose of grasping or acquiring a region of tissue to be formed into a plication. Tissue acquisition member 28 may extend distally from handle 16 through body 12 and distally between upper bail 20 and lower bail 26. Acquisition member 28 may also be translatable and rotatable within body 12 such that tissue engager 30 is able to translate longitudinally between upper bail 20 and lower bail 26. To support the longitudinal and rotational movement of acquisition member 28, an optional guide or linear bearing 32 may be connected to upper 20 or lower bail 26 to freely slide thereon. Guide 32 may also be slidably connected to acquisition member 28, such that guide 32 supports the longitudinal motion of acquisition member 28.

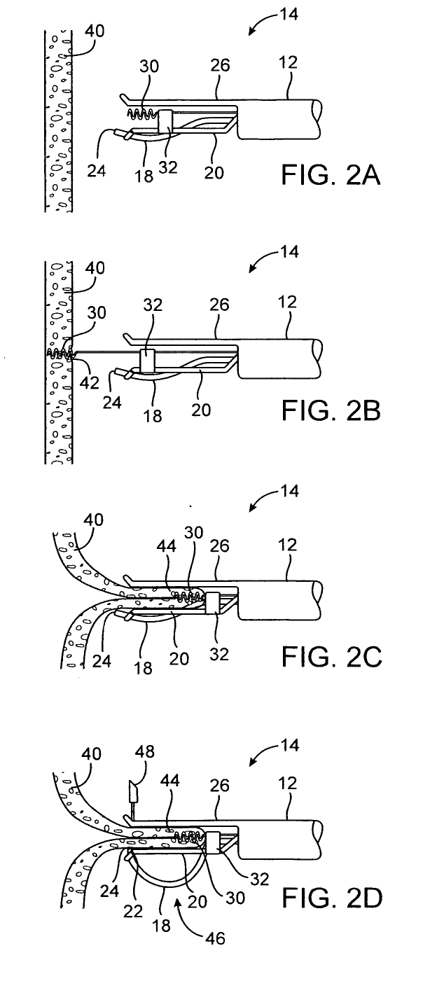

An example of a tissue plication procedure is seen in FIGS. 2A to 2D for delivering and placing a tissue anchor and is disclosed in further detail in co-pending U.S. patent application Ser. No. 10/840,950 filed May 7, 2004, which has been incorporated by reference above. Tissue manipulation assembly 14, as seen in FIG. 2A, may be advanced into a body lumen such as the stomach and positioned adjacent to a region of tissue wall 40 to be plicated. During advancement, launch tube 18 may be configured in a delivery profile such that tube 18 is disposed within or between the arms of upper bail 20 to present a relatively small profile.

Once tissue manipulation assembly 14 has been desirably positioned relative to tissue wall 40, tissue grasper or engager 30 may be advanced distally such that tissue grasper or engager 30 comes into contact with tissue wall 40 at acquisition location or point 42. As tissue grasper or engager 30 is distally advanced relative to body 12, guide 32, if utilized, may slide distally along with tissue grasper or engager 30 to aid in stabilizing the grasper. If a helically-shaped tissue grasper or engager 30 is utilized, as illustrated in FIG. 2B, it may be rotated from its proximal end at handle 16 and advanced distally until the tissue at point 42 has been firmly engaged by tissue grasper or engager 30. This may require advancement of tissue grasper or engager 30 through the mucosal layer and at least into or through the underlying muscularis layer and possibly into or through the serosa layer.

The grasped tissue may then be pulled proximally between upper 20 and lower bails 26 via tissue grasper or engager 30 such that the acquired tissue is drawn into a tissue fold 44, as seen in FIG. 2C. As tissue grasper or engager 30 is withdrawn proximally relative to body 12, guide 32 may also slide proximally to aid in stabilizing the device especially when drawing the tissue fold 44.

Once the tissue fold 44 has been formed, launch tube 18 may be advanced from its proximal end at handle 16 such that a portion 46 of launch tube 18, which extends distally from body 12, is forced to rotate at hinge or pivot 22 and reconfigure itself such that portion 46 forms a curved or arcuate shape that positions launch tube opening 24 perpendicularly relative to a longitudinal axis of body 12 and/or bail members 20, 26. Launch tube 18, or at least portion 46 of launch tube 18, is preferably fabricated from a highly flexible material or it may be fabricated, e.g., from Nitinol tubing material which is adapted to flex, e.g., via circumferential slots, to permit bending. Alternatively, assembly 14 may be configured such that launch tube 18 is reconfigured simultaneously with the proximal withdrawal of tissue grasper or engager 30 and acquired tissue 44.

As discussed above, the tissue wall of a body lumen, such as the stomach, typically comprises an inner mucosal layer, connective tissue, the muscularis layer and the serosa layer. To obtain a durable purchase, e.g., in performing a stomach reduction procedure, the staples or anchors used to achieve reduction of the body lumen are preferably engaged at least through or at the muscularis tissue layer, and more preferably, the serosa layer. Advantageously, stretching of tissue fold 44 between bail members 20, 26 permits an anchor to be ejected through both the muscularis and serosa layers, thus enabling durable gastrointestinal tissue approximation.

As shown in FIG. 2D, once launch tube opening 24 has been desirably positioned relative to the tissue fold 44, needle assembly 48 may be advanced through launch tube 18 via manipulation from its proximal end at handle 16 to pierce preferably through a dual serosa layer through tissue fold 44. Needle assembly 48 is preferably a hollow tubular needle through which one or several tissue anchors may be delivered through and ejected from in securing the tissue fold 44, as further described below.

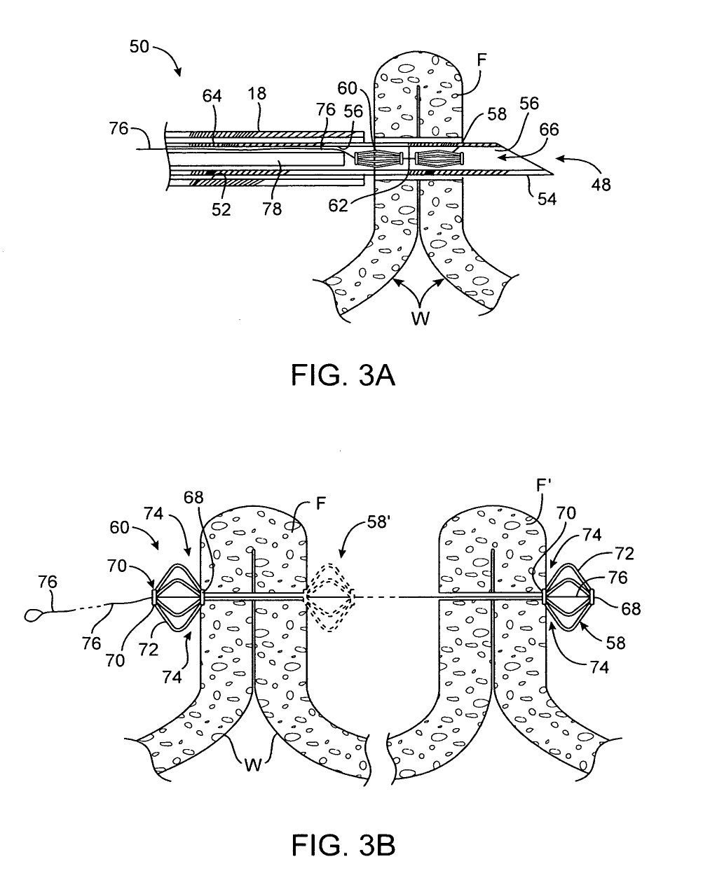

Because needle assembly 48 penetrates the tissue wall twice, it exits within the body lumen, thus reducing the potential for injury to surrounding organs. A detail cross-sectional view is shown in FIG. 3A of anchor delivery assembly 50 in proximity to tissue fold F. In this example, tissue fold F may comprise a plication of tissue created using the apparatus described herein or any other tool configured to create such a tissue plication. Tissue fold F 30 may be disposed within a gastrointestinal lumen, such as the stomach, where tissue wall W may define the outer or serosal layer of the stomach. Anchor delivery assembly may generally comprise launch tube 18 and needle assembly 48 slidingly disposed within launch tube lumen 52. Needle assembly 48 is generally comprised of needle 54, which is preferably a hollow needle having a tapered or sharpened distal end to facilitate its travel into and/or through the tissue. Other parts of the assembly, such as upper and lower bail members 20, 26, respectively, and tissue acquisition member 28 have been omitted from these figures only for clarity.

Once launch tube 18 has been desirably positioned with respect to tissue fold F, needle 54 may be urged or pushed into or through tissue fold F via delivery push tube or catheter 64 from its proximal end preferably located within handle 16. Delivery push tube or catheter 64 may comprise an elongate flexible tubular member to which needle 54 is connected or attached via joint 62. Alternatively, needle 54 and delivery push tube 64 may be integrally formed from a singular tubular member. Needle 54 may define needle lumen 56 through which basket anchor assembly 66, i.e., distal anchor 58 and/or proximal anchor 60 may be situated during deployment and positioning of the assembly. A single suture or flexible element 76 (or multiple suture elements) may connect proximal anchor 60 and distal anchor 58 to one another. For instance, element 76 may comprise various materials such as monofilament, multifilament, or any other conventional suture material, elastic or elastomeric materials, e.g., rubber, biocompatible metal wire, such as Nitinol, stainless steel, Titanium, etc.

The proximal end of suture 76 may pass slidingly through proximal anchor 60 to terminate in a suture loop. The proximal end of suture 76 may terminate proximally of the apparatus 10 within control handle 16, proximally of control handle 16, or at some point distally of control handle 16. In this variation, a suture loop may be provided to allow for a grasping or hooking tool to temporarily hold the suture loop for facilitating the cinching of proximal 60 and distal 58 anchors towards one another for retaining a configuration of tissue fold F, as described in further detail in U.S. patent application Ser. No. 10/840,950, which has been incorporated by reference above.

After needle assembly 48 has been pushed distally out through launch tube opening 24 and penetrated into and/or through tissue fold F, as shown in FIG. 3A, anchor pushrod or member 78 may be actuated also via its proximal end to eject distal anchor 58. Once distal anchor 58 has been ejected distally of tissue fold F, needle 54 may be retracted back through tissue fold F by either retracting needle 54 back within launch tube lumen 18 or by withdrawing the entire anchor delivery assembly 50 proximally relative to tissue fold F.

Once needle 54 has been retracted, proximal anchor 60 may then be ejected from launch tube 18 on a proximal side of tissue fold F. With both anchors 58, 60 disposed externally of launch tube 18 and suture 76 connecting the two, proximal anchor 60 may be urged into contact against tissue fold F, as shown in FIG. 3B. As proximal anchor 60 is urged against tissue fold F, proximal anchor 60 or a portion of suture 76 may be configured to provide any number of directionally translatable locking mechanisms which provide for movement of an anchor along suture 76 in a first direction and preferably locks, inhibits, or prevents the reverse movement of the anchor back along suture 76. In other alternatives, the anchors may simply be delivered through various elongate hollow tubular members, e.g., a catheter, trocars, etc.

The basket anchors may comprise various configurations suitable for implantation within a body lumen. Basket anchors are preferably reconfigurable from a low profile delivery configuration to a radially expanded deployment configuration in which a number of struts, arms, or mesh elements may radially extend once released from launch tube 18 or needle 54. Materials having shape memory or superelastic characteristics or which are biased to reconfigure when unconstrained are preferably used, e.g., spring stainless steels, Ni—Ti alloys such as Nitinol, etc. In FIGS. 3A and 3B, each of the basket anchor 58, 60 is illustrated as having a number of reconfigurable struts or arm members 72 extending between distal collar 68 and proximal collar 70; however, this is intended only to be illustrative and suitable basket anchors are not intended to be limited to baskets only having struts or arms. Examples of suitable anchors are further described in detail in U.S. patent application Ser. No. 10/612,170, which has already been incorporated herein above.

FIG. 3B shows distal basket anchor 58 delivered through tissue fold F via needle 54 and launch tube 18. As above, the other parts of the plication assembly, such as upper and lower bail members 20, 26, respectively, and tissue acquisition member 28 have been omitted from these figures only for clarity.

FIG. 3B shows one variation where a single fold F may be secured between proximal anchor 60 and distal anchor 58′. As seen, basket anchor 58′ has been urged or ejected from needle 54 and is shown in its radially expanded profile for placement against the tissue surface. In such a case, a terminal end of suture 76 may be anchored within the distal collar of anchor 58′ and routed through tissue fold F and through, or at least partially through, proximal anchor 60, where suture 76 may be cinched or locked proximally of, within, or at proximal anchor 60 via any number of cinching mechanisms. Proximal anchor 60 is also shown in a radially expanded profile contacting tissue fold F along tissue contact region 74. Locking or cinching of suture 76 proximally of proximal anchor 60 enables the adequate securement of tissue fold F.

Various examples of cinching devices and methods which may be utilized with the tools and devices herein are described in further detail in U.S. patent application Ser. No. 10/840,950 filed May 7, 2004, which has been incorporated herein above.

If additional tissue folds are plicated for securement, distal basket anchor 58 may be disposed distally of at least one additional tissue fold F′, as shown in FIG. 3B, while proximal anchor 60 may be disposed proximally of tissue fold F. As above, suture 76 may be similarly affixed within distal anchor 58 and routed through proximal anchor 60, where suture 76 may be cinched or locked via proximal anchor 60, as necessary. If tissue folds F and F′ are to be positioned into apposition with one another, distal basket anchor 58 and proximal anchor 60 may be approximated towards one another. As described above, proximal anchor 60 is preferably configured to allow suture 76 to pass freely therethrough during the anchor approximation. However, proximal anchor 60 is also preferably configured to prevent or inhibit the reverse translation of suture 76 through proximal anchor 60 by enabling uni-directional travel of anchor 60 over suture 76. This cinching feature thereby allows for the automated locking of anchors 58, 60 relative to one another during anchor approximation.

With respect to the anchor assemblies described herein, the types of anchors shown and described are intended to be illustrative and are not limited to the variations shown. For instance, several of the tissue anchor variations are shown as “T”-type anchors while other variations are shown as reconfigurable “basket”-type anchors, which may generally comprise a number of configurable struts or legs extending between at least two collars or support members. Other variations of these or other types of anchors are also contemplated for use in an anchor assembly. Moreover, a single type of anchor may be used exclusively in an anchor assembly; alternatively, a combination of different anchor types may be used in an anchor assembly. Furthermore, the different types of cinching or locking mechanisms are not intended to be limited to any of the particular variations shown and described but may be utilized in any of the combinations or varying types of anchors as practicable.

The upper and/or lower extension members or bails may also be configured into a variety of embodiments, which may be utilized in any number of combinations with any of the tissue acquisition member variations as practicable. Although the upper and lower extension members or bails may be maintained rigidly relative to one another, the upper and/or lower extension members may be alternatively configured to articulate from a closed to an open configuration or conversely from an open to a closed configuration for facilitating manipulation or stabilization of tissue drawn between the bail members.

In operation, once the selected region of tissue has been acquired by the tissue grasper 30, the obtained tissue may be proximally withdrawn between the bail members, which may act as stabilizers for the tissue. To accommodate large portions of grasped tissue between the bail members, one or both bail members may be articulated or urged to open apart from one another to allow the tissue to enter and become positioned between the bail members. One or both bail members may then be articulated or urged to clamp or squeeze the tissue fold between the bail members to facilitate stabilization of the tissue fold for tissue manipulation and/or anchor deployment and/or any other procedure to be undertaken.

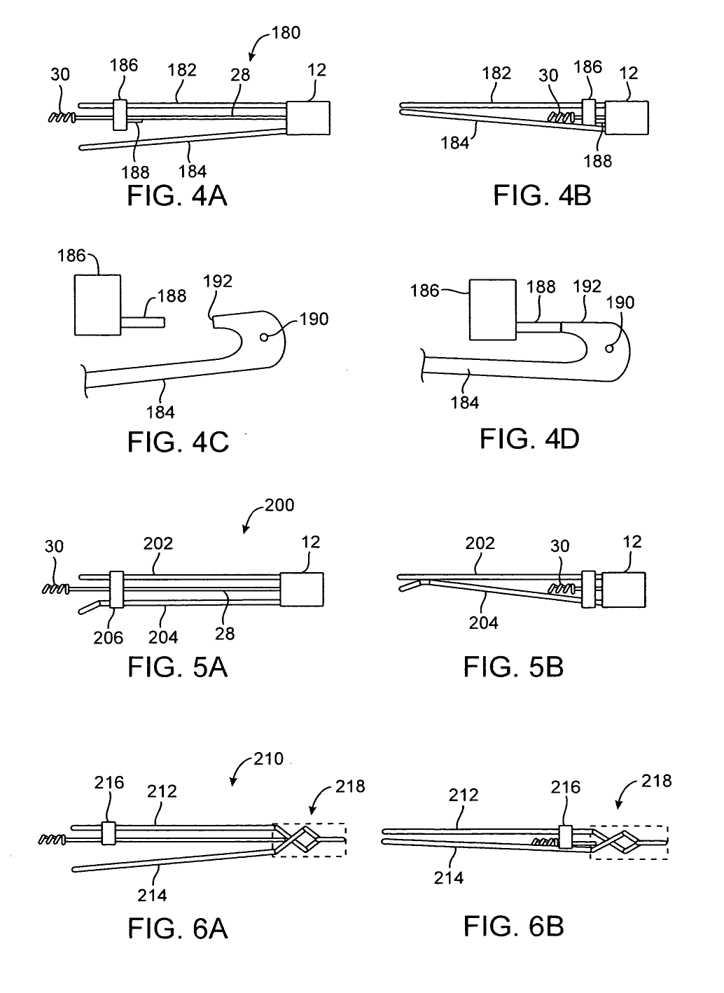

One such articulatable extension assembly may be seen in the side views of FIGS. 4A and 4B. Other features such as the launch tube and tubular body have been omitted merely for the sake of clarity for the following illustrations. As seen in FIG. 4A, upper extension member 182 and lower extension member 184 of active extension assembly 180 may be configured to have an open or spread configuration relative to one another when guide or linear bearing 186 is positioned distally along upper extension member 182. Linear bearing 186 may be configured to slide freely along upper extension member 182 when urged by acquisition member 28 distally or proximally. Rather than having linear bearing 186 slide along upper extension member 182, it may be configured alternatively to slide along lower extension member 184.

With tissue grasper 30 and acquisition member 28 distally protruding from extension members 182, 184, as shown in FIG. 4A, the desired region of tissue may be acquired by rotating tissue grasper 30 into the tissue. Once tissue has been acquired by tissue grasper 30, the tissue may be pulled between the opened extension members 182, 184 by proximally withdrawing tissue grasper 30 and linear bearing 186 may be forced proximally over upper extension member 182, as shown in the detail view of FIG. 4C. One or more projections or pistons 188 may protrude proximally from linear bearing 186 such that one or more of these projections 188 comes into contact with actuation lever or member 192, as shown in FIG. 4D, which may be located proximally of extension members 182, 184 and connected in a pivoting relationship with lower extension member 184 about pivot 190. As linear bearing 186 is urged proximally and projection 188 presses against actuation lever 192, lower extension member 184 may be rotated about pivot 190 such that lower extension member 184 is urged towards upper extension member 182 to securely clamp onto and retain any tissue positioned between the extension members 182, 184.

Another articulatable extension assembly may be seen in assembly 200 in the side views of FIGS. 5A and 5B. In this variation, upper extension member 202 may project distally opposite lower extension member 204 which may be biased to close towards upper extension member 202. When tissue grasper 30 is advanced to engage tissue, as shown in FIG. 5A, linear bearing 206 may be urged distally along upper extension member 202 via acquisition member 28 such that lower extension member 204 is forced or wedged away from upper extension member 202. Once the tissue is engaged and withdrawn proximally, linear bearing 206 may be pulled proximally while sliding along lower member 204 and allowing lower member 204 to spring back towards upper member 202 and over any tissue positioned therebetween, as shown in FIG. 5B.

Another articulatable extension assembly is shown in the side views of extension assembly 210 of FIGS. 6A and 6B. In this variation, upper extension member 212 and/or lower extension member 214 may be connected to linkage assembly 218 located proximally of the extension members 212, 214. Linkage assembly 218 may be manipulated via any number of control mechanisms such as control wires to urge extension members 212, 214 between open and closed configurations. Alternatively, linkage assembly 218 may be configured to open or close upon the proximal or distal advancement of linear bearing 216 relative to linkage assembly.

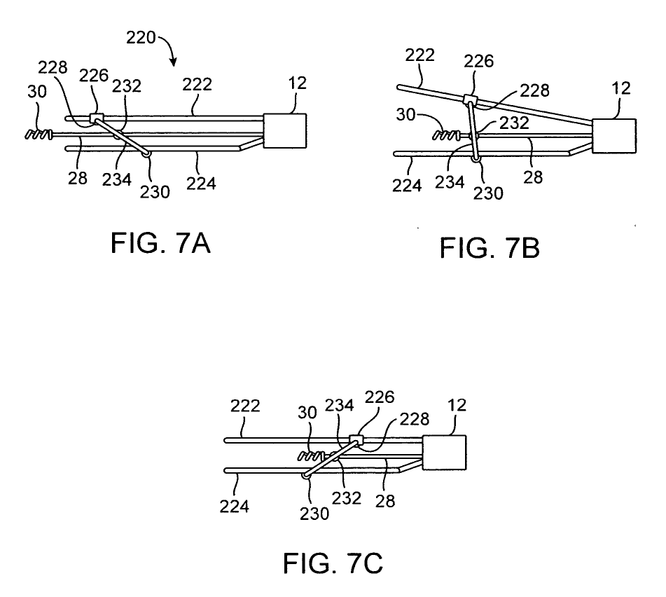

FIGS. 7A to 7C show side views of another variation in extension assembly 220 where upper and lower extension members 222, 224 are articulatable between open and closed configurations via a pivoting arm or member 234 interconnecting the two. In this example, a first end of pivoting arm 234 may be in a pivoting connection at pivot 228 with linear bearing 226, which may slide translationally along upper member 222. A second end of pivoting arm 234 may also be in a pivoting connection with lower extension member 224 at pivot 230, which may remain fixed to lower member 224. Acquisition member 28 may also be in a third pivoting connection with pivoting arm 234 at pivot 232, which may also be configured to allow for the linear translation of acquisition member therethrough.

In operation, when acquisition member 28 and tissue grasper 30 is advanced distally, as shown in FIG. 7A, both upper and lower extension members 222, 224 are in a closed configuration with linear bearing 226 being advanced distally along upper extension member 222. As tissue grasper 30 is withdrawn proximally between extension members 222, 224, pivoting arm 234 may be pivoted about fixed pivot 230 on lower member 224 while upper member 222 is urged into an open configuration as linear bearing 226 is urged proximally over upper member 222, as shown in FIG. 7B. This expanded or open configuration allows for the positioning of large portions of tissue to be drawn between the extension members 222, 224 for stabilization. FIG. 7C shows tissue grasper 30 as having been further withdrawn and linear bearing 226 urged proximally such that upper member 222 is urged back into a closed configuration relative to lower member 224. The closing of extension members 222, 224 allows for the members to further clamp upon any tissue therebetween for further stabilization of the tissue.

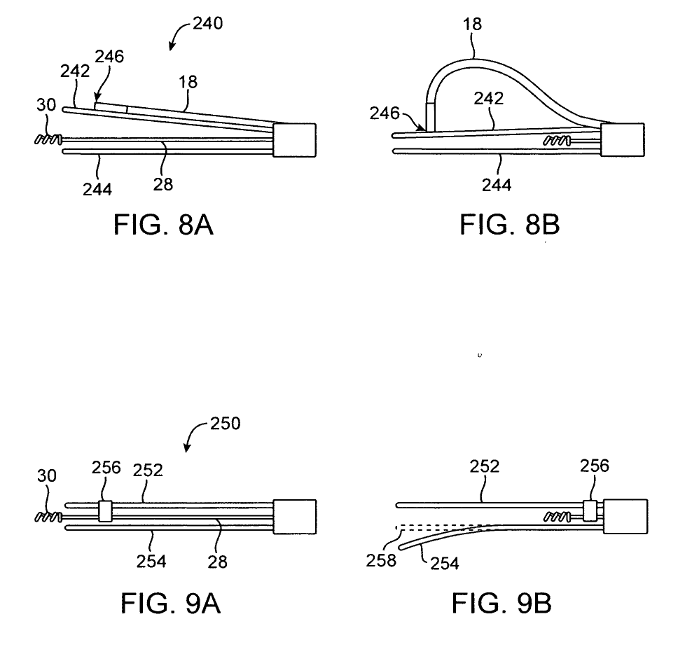

FIGS. 8A and 8B show another alternative in active extension assembly 240. In this variation, upper extension member 242 may be biased to extend away from lower extension member 244. As shown in FIG. 8A, upper extension member 242 may remain in an open configuration relative to lower member 244 for receiving tissue therebetween. In this variation, biased upper member 242 may be urged into a closed configuration by pivoting the launch tube 18 about pivot 246, which may be located along upper member 242. As launch tube 18 is pivoted into an anchor deployment configuration, the pivoting action may urge upper member 242 towards lower member 244 to clamp upon any tissue therebetween.

FIGS. 9A and 9B show yet another alternative in assembly 250 where upper extension member 252 and/or lower extension member 254 may be passively urged into an open configuration. In this example, lower extension member 254 is shown as being flexed from a relaxed configuration in FIG. 9A to a flexed configuration in FIG. 9B. As linear bearing 256 is withdrawn proximally, any tissue engaged to tissue grasper 30 may urge lower extension member 254 from its normal position 258 to its flexed and opened position. Accordingly, lower extension member 254 and/or upper extension member 252 may be made from a relatively flexible plastic or metallic material, e.g., Nitinol, spring stainless steel, etc. When tissue is removed from between the extension members 252, 254, lower extension member 254 may return to its normal configuration 258.

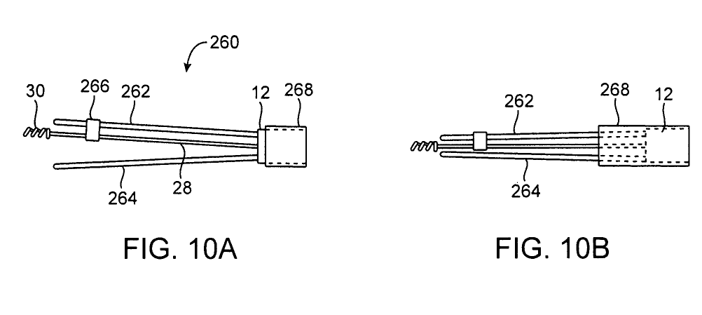

FIGS. 10A and 10B show side views of another assembly 260 in which upper and/or lower extension members 262, 264 may be biased or configured to flex away from one another, as shown in FIG. 10A. Once linear bearing 266 and tissue grasper 30 has been retracted, an outer sleeve 268 slidingly disposed over tubular body 12 may be pushed distally such that sleeve 268 is slid over at least a proximal portion of extension members 262, 264 such that they are urged towards one another into a closed configuration onto tissue which may be present therebetween, as shown in FIG. 10B.

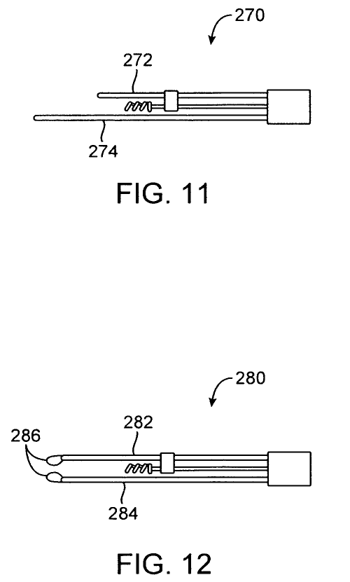

Aside from features such as articulation of the extension members, the extension members themselves may be modified. For instance, FIG. 11 shows a side view of extension assembly 270 where lower extension member 274 may be extended in length relative to upper extension member 272. The length of lower extension member 274 may be varied depending upon the desired result. Alternatively, upper extension member 272 may be shortened relative to lower extension member 274. The lengthening of lower extension member 274 may be utilized to present a more stable platform for tissue approximated between the extension members 262, 264.

Another alternative for modifying the extension members is seen in the side view of FIG. 12 in extension assembly 280. In this example, one or both extension members 282, 284 may be configured to have atraumatic blunted ends 286 which may be further configured to be flexible to allow tissue to slide over the ends. Moreover, atraumatic ends 286 may be configured in a variety of ways provided that an atraumatic surface or feature is presented to the tissue.

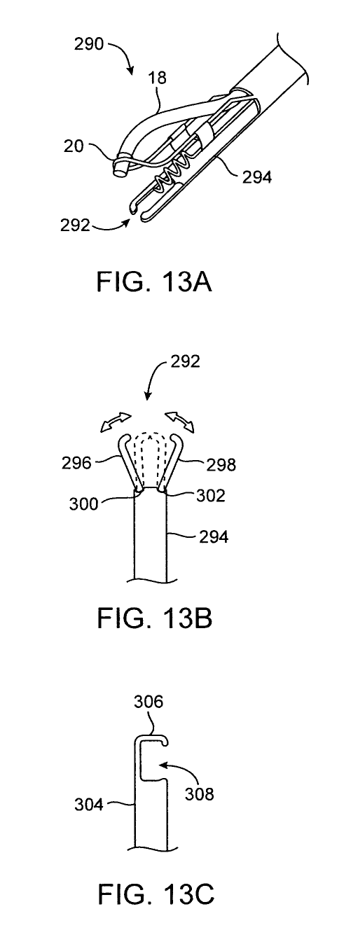

In addition to atraumatic features, the lower extension member of the tissue manipulation assembly may be varied as well. For example, as the needle assembly and tissue anchors are deployed from the launch tube, typically from the upper extension member, it is preferable to have sufficient clearance with respect to the lower extension member so that unhindered deployment is facilitated. One method for ensuring unhindered deployment is via a lower extension member having a split opening defined near or at its distal end, as shown in the perspective view of tissue manipulation assembly 290 in FIG. 13A. Such a split may allow for any deployed anchors or suture an opening through which to be released from assembly 290.

Additionally, the jaws that define the opening may be articulatable as well relative to lower extension member 294. As shown in the bottom view of FIG. 13B, articulatable lower extension assembly 292 may have one or both jaw members 296, 298 articulatable via pivots 300, 302, respectively, relative to lower extension member 294 such that one or both jaw members 296, 298 are able to be moved between a closed configuration, as shown in FIG. 13A, and an open configuration, as shown in FIG. 13B. This variation in assembly 290 may allow for any needle or anchor assemblies to easily clear lower extension member 294.

Another variation of lower extension member 304 is shown in the bottom view of FIG. 13C. In this variation, an enclosing jaw member 306 may extend from lower extension member 304 such that an opening 308 along either side of extension member 304 is created. Such an opening 308 may create a “C”-shaped lower extension member 304 which may facilitate needle and anchor deployment from the tissue manipulation assembly.

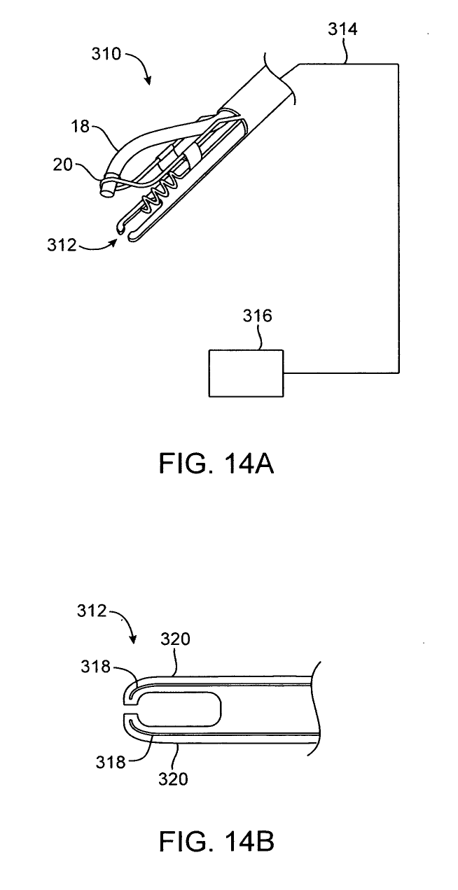

Another variation of a tissue manipulation assembly 310 may be seen in the illustrative partial perspective view of FIG. 14A. In addition to articulation or release features, one or both extension members may be utilized to selectively ablate regions of tissue. Assembly 310 for instance may have a tissue ablation assembly 312 integrated into the lower extension member 320. Such a tissue ablation assembly 312, as seen in the top view of FIG. 14B, may incorporate one or more wires or electrically conductive elements 318 upon lower extension member 320 to create a tissue ablation region. The lower extension member 320 may be fabricated from a non-conductive material upon which wires 318 may be integrated. Alternatively, the entire lower member 320 may be electrically conductive with regions selectively insulated leaving non-insulated areas to create ablation regions 318. The wires or regions 318 may be electrically connected via wires 314 to power source 316, which may provide various forms of energy for tissue ablation, e.g., radio-frequency, microwave, etc.

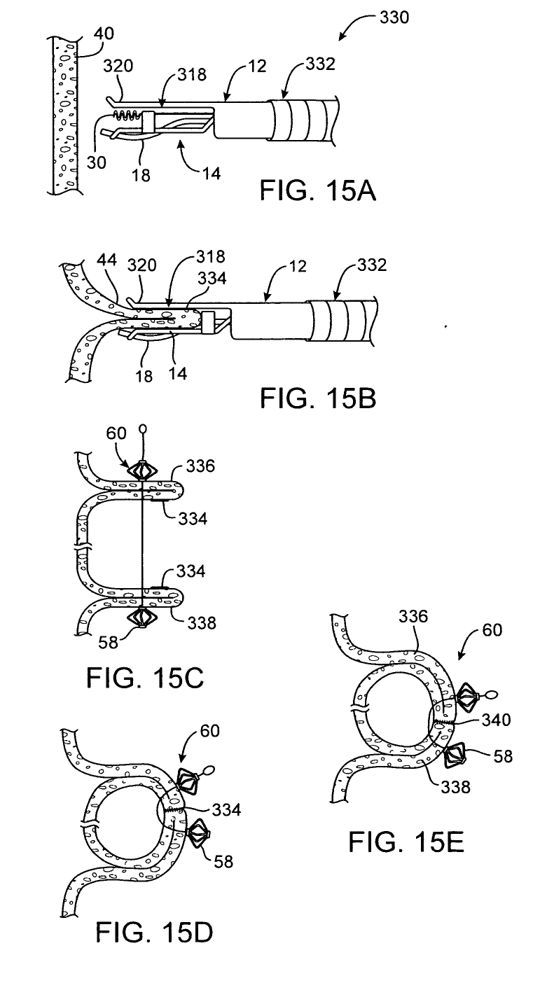

One example for use of the ablative tissue manipulation assembly may be seen in FIGS. 15A to 15E where tissue approximation assembly 330 may be seen with tissue manipulation assembly 14 advanced through an optional shape-lockable overtube 332. Ablation region 318 is integrated into the lower extension member 320 of the tissue manipulation assembly, as above. Alternatively, region 318 may, for example, comprise an abrasive surface disposed on lower extension member 320. Alternatively, the lower extension member 320 may comprise an ablation electrode for injuring mucosal tissue.

As seen in FIG. 15B, when tissue wall 40 is folded between the extension members of assembly 14, target mucosal tissue 334 contacts lower extension member 320 as well as ablation region 318. Passive or active actuation of ablation region 318 may then injure and/or remove the target mucosal tissue 334. As further seen in FIG. 15C, this procedure may be repeated at one or more additional tissue folds 336, 338 that may then be approximated together, as in FIG. 15D. The contacting injured regions of mucosal tissue promote healing and fusion 340 of the approximated folds, as in FIG. 15E.

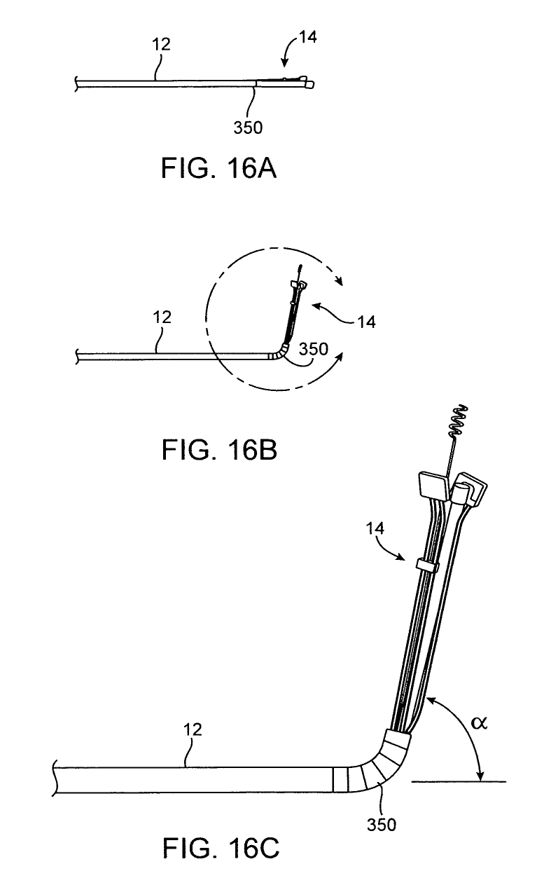

Aside from variations on aspects of the tissue manipulation assembly, the entire assembly may also be modified to adjust the tissue manipulation assembly position relative to the tubular body upon which the assembly is attachable. FIG. 16A shows a distal portion of tubular body 12 and tissue manipulation assembly 14 connected thereto. While tubular body 12 may comprise a rigid or flexible length, tissue manipulation assembly 14 may be further configured to articulate relative to tubular body 12, as shown in FIG. 16B, to further enhance the maneuverability and manipulation capabilities of tissue manipulation assembly 14. In one example, assembly 14 may be connected to tubular body 12 via a hinged or segmented articulatable portion 350, shown in the detail FIG. 16C, which allows assembly 14 to be reconfigured from a low-profile configuration straightened relative to tubular body 12 to an articulated configuration where assembly 14 forms an angle, α, relative to tubular body 12. The angle, α, may range anywhere from 180° to −180° depending upon the desired level of articulation. Articulatable portion 350 may be configured to allow assembly 14 to become articulated in a single plane or it may also be configured to allow a full range of motion unconstrained to a single plane relative to tubular body 12. Articulation of assembly 14 may be accomplished any number of various methods, e.g., control wires.

The tissue manipulation assembly may be manipulated and articulated through various mechanisms. One such assembly that integrates each of the functions into a singular unit may be seen in the handle assembly 16, which is connected via tubular body 12 to the tissue manipulation assembly. Such a handle assembly may be configured to separate from tubular body 12, thus allowing for reusability of the handle. Moreover, such a handle may be fabricated from a variety of materials such as metals or plastics, provided that the materials are preferably biocompatible. Examples of suitable materials may include stainless steel, PTFE, Delrin®, etc.

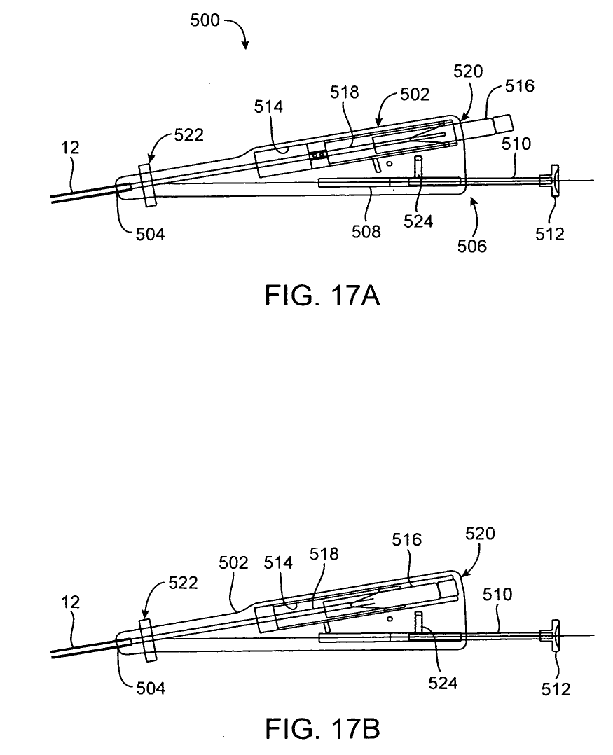

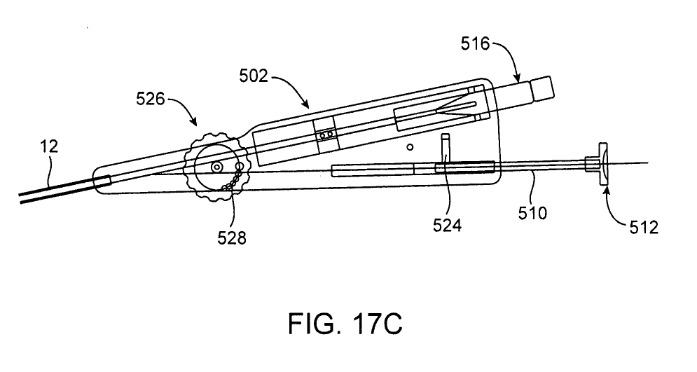

One variation of a handle assembly 16 is shown in the illustrative side view of handle 500 in FIG. 17A with half of handle enclosure 502 removed for clarity for discussion purposes. As shown, handle enclosure 502 may connect with tubular body 12 at its distal end at tubular interface 504. The proximal end of handle 500 may define acquisition member opening 506 which opens to acquisition member receiving channel 508 defined through enclosure 502 from opening 506 to tubular interface 504. The acquisition member 28 may be routed through receiving channel 508 with the proximal end 510 of acquisition member 28 extending proximally of enclosure 502 for manipulation by the user. Acquisition member proximal end 510 may further have an acquisition member rotational control 512 that the user may grasp to manipulate acquisition member 28.

Acquisition member receiving channel 508 preferably has a diameter which is sufficiently large enough to allow for the translational and rotational movement of acquisition member through the receiving channel 508 during tissue manipulation. Acquisition member lock 524, e.g., a screw or protrusion, may also extend at least partially into acquisition member receiving channel 508 such that lock 524 may be urged selectively against acquisition member 28 to freeze a position of acquisition member 28, if so desired. The terminal end of receiving channel 508 may extend to tubular interface 504 such that receiving channel 508 and tubular body 12 are in communication to provide for the passage of acquisition member 28 therethrough.

In addition to the acquisition member controls, the handle enclosure 502 may also provide a needle assembly receiving channel 514 through which needle assembly control 516 and needle assembly catheter 518 may be translated through. Needle assembly receiving channel 514 may extend from needle assembly opening 520 also to tubular interface 504. Needle assembly receiving channel 514 extends to tubular interface 504 such that needle assembly receiving channel 514 and tubular body 12 are also in communication to provide for the passage of needle assembly catheter 518 therethrough.

In operation, once the tissue to be plicated has been acquired and drawn between the lower and upper extension members by acquisition member 28, as described above, the launch tube 18 may be advanced distally and rotated into its deployment configuration. Once positioned for deployment, the needle assembly may be advanced into and/or through the tissue by urging needle assembly control 516 and needle assembly catheter 518 distally into needle assembly receiving channel 514, as shown by the advancement of control 516 in FIG. 17B. The tissue anchors may then be deployed from the needle assembly catheter 518 via the needle assembly control 516, as further described below. Withdrawal of the needle assembly from the tissue may be accomplished by the proximal withdrawal of needle assembly control 516 and assembly catheter 518.

Tissue manipulation articulation control 522 may also be positioned on handle 500 to provide for selective articulation of the tissue manipulation assembly, as shown above in FIGS. 16A to 16C. This variation shows articulation control 522 rotatably positioned on handle enclosure 502 such that articulation control 522 may be rotated relative to handle 500 to selectively control the movement of the tissue manipulation assembly. Articulation control 522 may be operably connected via one or several control wires attached between articulation control 522 and the tissue manipulation assembly. The control wires may be routed through tubular interface 504 and extend through tubular body 12.

FIG. 17C shows another variation of handle enclosure 502 where the tissue manipulation articulation control 526 may be positioned on a side surface of handle enclosure 502. Articulation control 526 may include a ratcheting mechanism 528 within enclosure 502 to provide for controlled articulation of the tissue manipulation assembly.

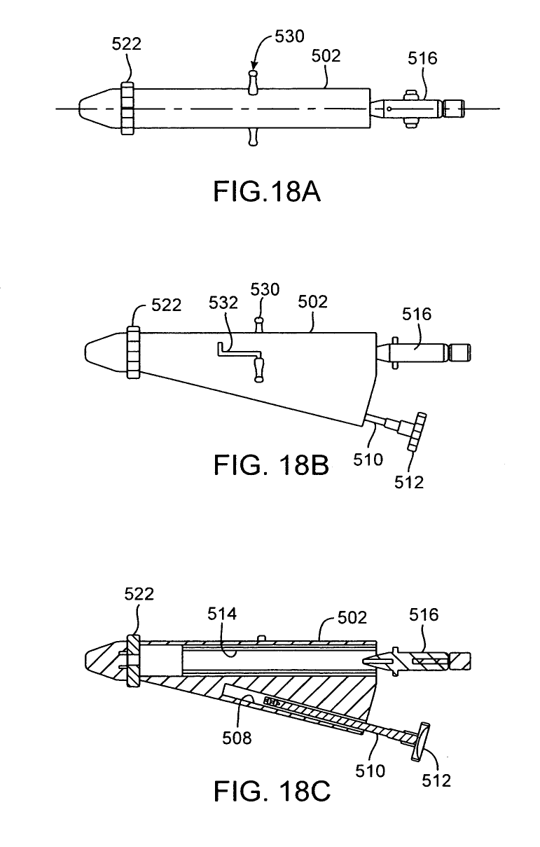

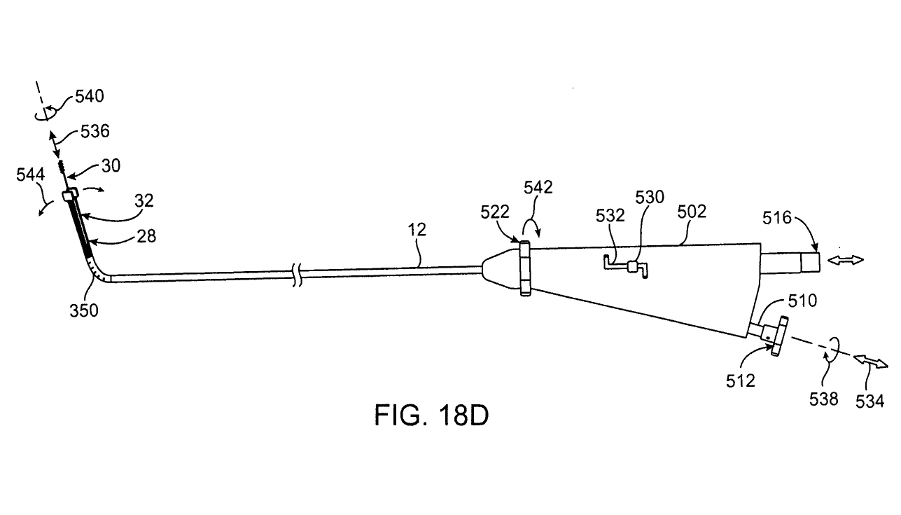

FIGS. 18A to 18C show top, side, and cross-sectional views, respectively, of another variation on the handle assembly. As seen in FIGS. 18A and 18B, an advancement control 530 may be adapted to selectively slide translationally and rotationally through a defined advancement channel or groove 532 defined within handle enclosure 502. Advancement control 530 may be used to control the deployment and advancement of needle assembly control 516 as well as deployment of the launch tube, as described in further detail below.

FIG. 18D shows an assembly side view of the handle assembly, tubular body 12, and tissue manipulation assembly and the corresponding motion of the assembly when manipulated by the handle. As described above, tissue acquisition member proximal end 510 and acquisition member control 512 may be advanced or withdrawn from the handle enclosure 502 in the direction of arrow 534 to transmit the corresponding translational motion through tubular body 12 to tissue acquisition member 28 and tissue grasper 30, as indicated by the direction of corresponding arrow 536. Likewise, when acquisition member control 512 is rotated relative to handle enclosure 502, as indicated by rotational arrow 538, the corresponding rotational motion is transmitted through tubular body 12 to tissue grasper 30 for screwing into or unscrewing from tissue, as indicated by corresponding rotational arrow 540. As mentioned above, tubular body 12 may be rigid or flexible depending upon the application utilized for the device.

Likewise, longitudinal translation of needle assembly control 516 relative to enclosure 502, as indicated by the arrow may transmit the corresponding longitudinal motion to the needle assembly through the launch tube when reconfigured for deployment. The tissue manipulation assembly articulation control 522 may also be seen in this handle variation as being rotatable in the direction of arrow 542 relative to handle enclosure 502. Depending upon the direction of articulation, control 522 may be manipulated to elicit a corresponding motion from the tissue manipulation assembly about hinge or articulatable section 350 in the direction of arrows 544.

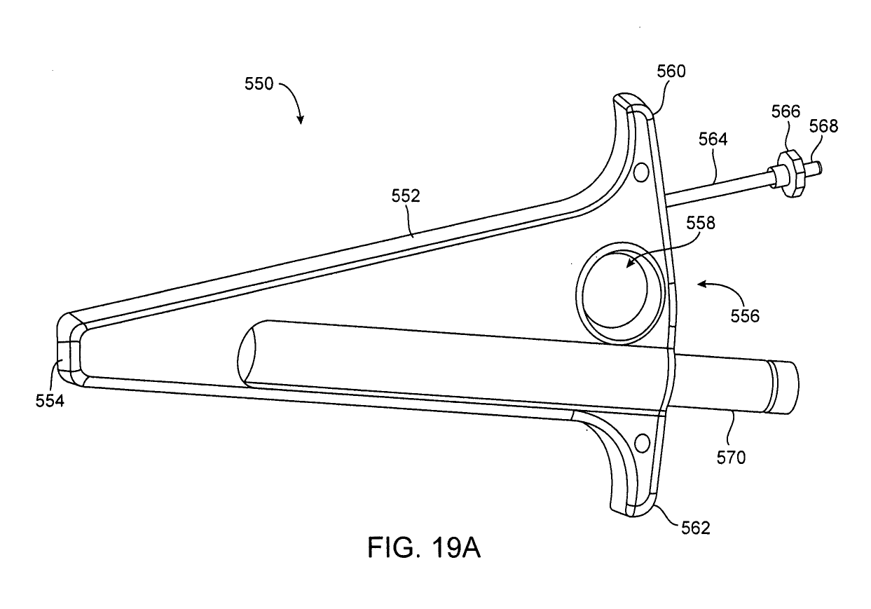

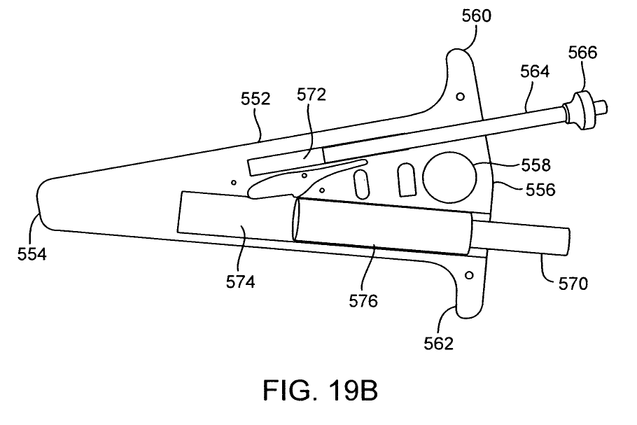

Another handle variation may be seen in the perspective view of handle assembly 550, as shown in FIG. 19A. This particular variation may have handle enclosure 552 formed in a tapered configuration which allows for the assembly 550 to be generally symmetrically-shaped about a longitudinal axis extending from its distal end 554 to its proximal end 556. The symmetric feature of handle assembly 550 may allow for the handle to be easily manipulated by the user regardless of the orientation of the handle enclosure 552 during a tissue manipulation procedure. An additional feature which may further facilitate the ergonomic usability of handle assembly 550 may further include at least one opening 558 defined through the enclosure 552 to allow the user to more easily grip and control the handle 550. Another feature may include grips 560, 562 which may extend from either side of enclosure 552.

As seen in the figure, acquisition member 564 may include additional features to facilitate control of the tissue. For instance, in this variation, in addition to the rotational control 566, an additional rotational control 568 may extend proximally from control 566 and have a diameter smaller than that of control 566 for controlling fine rotational motion of acquisition member 564.

FIG. 19B shows a side view of the handle assembly 550 of FIG. 19A with the enclosure 552 partially removed for clarity. As shown, needle assembly control 570 may be seen inserted within an additional needle deployment mechanism 576, as described below in further detail, within needle assembly receiving channel 574. Acquisition member 564 may also be seen positioned within acquisition member receiving channel 572.

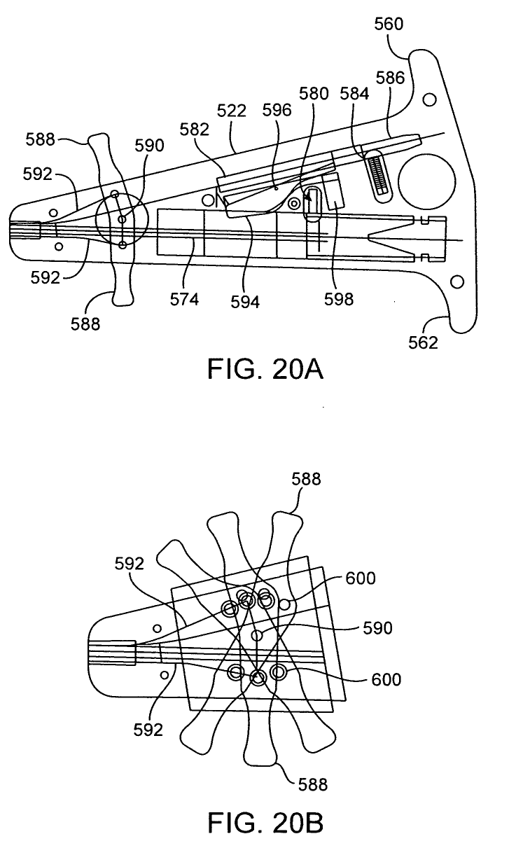

Yet another variation of the handle assembly may be seen in the side view of the handle assembly of FIG. 20A where the handle enclosure 522 is partially removed for clarity. In this variation, needle deployment mechanism lock 580, e.g., a screw or protrusion, may be configured to operably extend at least partially into needle assembly receiving channel 574 to selectively lock the launch tube and/or needle assembly control within receiving channel 574. Also shown is acquisition member receiving channel 582 through which the acquisition member may be translated and/or rotated. Acquisition member lock 584 may also be seen to extend at least partially into the acquisition member receiving channel 582 to selectively lock the acquisition member position, if so desired. The acquisition member receiving channel 582 may be optionally threaded 586 such that the acquisition member may be advanced or withdrawn using a screw-like mechanism.

An additional needle deployment mechanism lock 594 may also be seen pivotally mounted about pivot 596 within enclosure 522. Mechanism 594 may be biased via deployment mechanism biasing element 598, e.g., a spring, to maintain a biasing force against mechanism 594 such that the needle assembly control may automatically become locked during advancement within enclosure 522 to allow for a more controlled anchor deployment and needle assembly advancement.

Moreover, one or more pivotable tissue manipulation assembly controls 588 may be mounted to enclosure 522 and extend from one or both sides of enclosure 522 to provide for articulation control of the tissue manipulation assembly, as described above. As presently shown in FIG. 20B in the detail side view from the handle assembly of FIG. 20A, one or more control wires 592 may be connected to control 588 at control wire attachment points 600. Control 588 may pivot about tissue acquisition pivot 590 located within handle enclosure 522. As control 588 is pivoted, the articulation of control wires 592 may articulate a position of the tissue manipulation assembly, as discussed above. FIG. 20B shows an example of the range of motion which may be possible for control 588 as it is rotated about pivot 590.

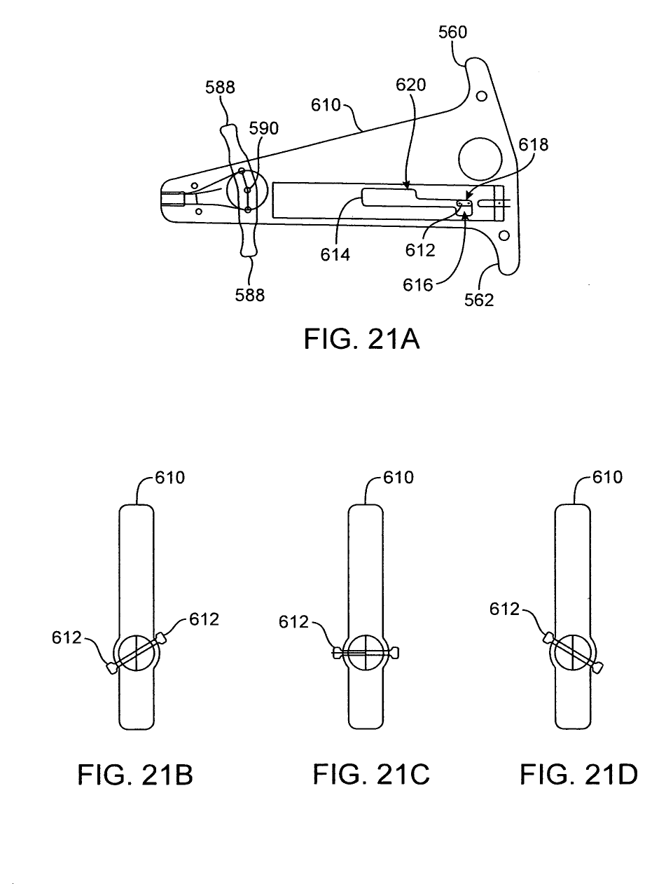

FIG. 21A shows a side view of another variation of handle enclosure 610 which incorporates a needle deployment locking and advancement control 612 which is adapted to be advanced and rotated within needle deployment travel 614 into various positions corresponding to various actions. Locking control 612 may be utilized in this variation to selectively control access of the needle assembly within handle enclosure 610 as well as deployment of the needle assembly and launch tube advancement with a single mechanism. A needle assembly, such as needle assembly 570, may be advanced into handle enclosure 610 with locking control 612 initially moved into needle assembly receiving position 616, shown also in the end view of FIG. 21B. Once the needle assembly has been initially introduced into enclosure 610, the needle assembly may be locked within enclosure 610 by rotating locking control 612 into its needle assembly locking position 618, clockwise rotation as shown in the end view of FIG. 21C. The needle assembly may be locked within enclosure 610 to prevent the accidental withdrawal of the needle assembly from the enclosure 610 or inadvertent advancement of the needle assembly into the tissue.

With locking control 612 in the needle assembly locking position 618, the needle deployment mechanism within enclosure 610 may also be longitudinally translated in a distal direction by urging locking control 612 distally within needle deployment travel 614. Urging locking control 612 distally translates not only the needle deployment mechanism within enclosure 610, but may also translate the launch tube distally such that the launch tube distal portion is pivoted into its deployment configuration, as described above. As the needle deployment mechanism is distally translated within enclosure 610, the needle assembly may also be urged distally with the deployment mechanism such that needle assembly becomes positioned within the launch tube for advancing the needle body into the tissue.

Once locking control 612 has been advanced distally, locking control 612 may again be rotated into the needle assembly release position 620, clockwise rotation as shown in the end view of FIG. 21D. Once in the release position 620, the needle assembly may be free to be translated distally within enclosure 610 for advancing the needle assembly and needle body relative to the launch tube and enclosure 610. To remove the needle assembly from enclosure 610, the steps may be reversed by moving locking control 612 proximally back to its initial needle assembly receiving position 616 so that the needle assembly is unlocked from within enclosure 610. A new needle assembly may then be introduced into enclosure 610 and the process repeated as many times as desired.

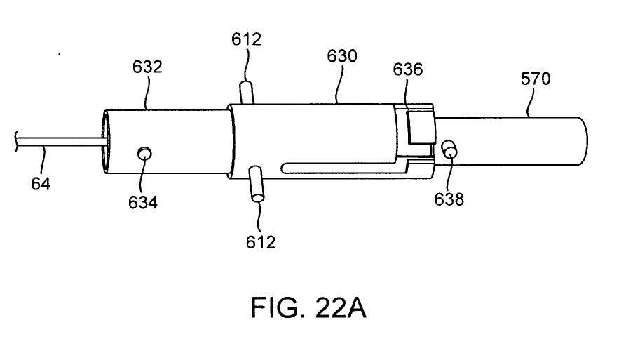

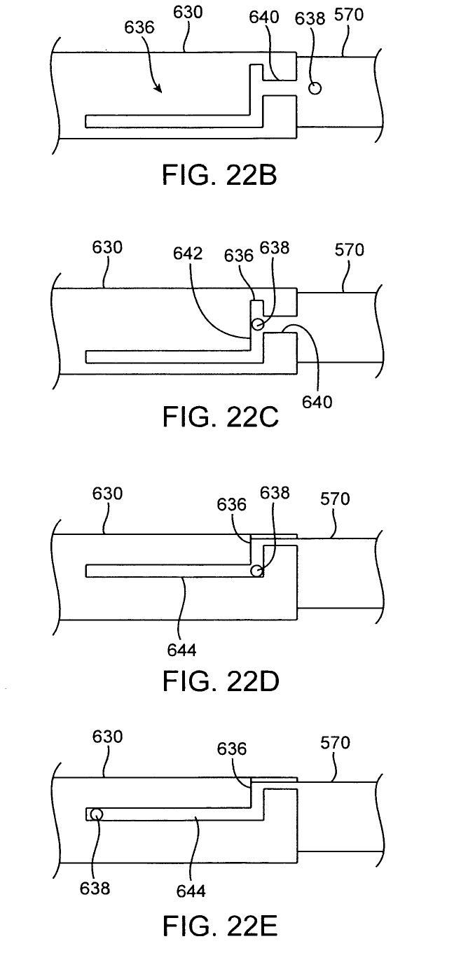

Details of one variation of the locking mechanism disposed within the handle enclosure 610 are shown in the perspective view of FIG. 22A. The other elements of the handle assembly have been omitted from this illustration for clarity. The locking mechanism may generally be comprised of outer sleeve 630 disposed about inner sleeve 632. Outer sleeve 630 preferably has a diameter which allows for its unhindered rotational and longitudinal movement relative to inner sleeve 632. Needle deployment locking control 612 may extend radially from outer sleeve 630 and protrude externally from enclosure 610, as described above, for manipulation by the user. Outer sleeve 630 may also define needle assembly travel path 636 along its length. Travel path 636 may define the path through which needle assembly 570 may traverse in order to be deployed. Needle assembly 570 may define one or more guides 638 protruding from the surface of assembly 570 which may be configured to traverse within travel path 636. Inner sleeve 634 may also define guides 634 protruding from the surface of inner sleeve 634 for traversal within grooves defined in handle enclosure 610. Moreover, outer sleeve 630 is preferably disposed rotatably about inner sleeve 632 such that outer sleeve 630 and inner sleeve 632 are configured to selectively interlock with one another in a corresponding manner when locking control 612 is manipulated into specified positions.

Turning to FIGS. 22B to 22E, the operation of the locking mechanism of FIG. 22A is described in further detail. As needle assembly 570 is initially introduced into handle enclosure 610 and the locking mechanism, needle assembly 570 may be rotated until guides 638 are able to slide into longitudinal receiving channel 640 of travel path 636 defined in outer sleeve 630, as shown in FIGS. 22B and 22C. Locking control 612 may be partially rotated, as described above in FIGS. 21B and 21C, such that outer sleeve is rotated with respect to needle assembly 570 and guides 638 slide through transverse loading channel 642, as shown in FIG. 22D. In this position, the locking mechanism may be advanced distally to deploy the launch tube and to also advance needle assembly 570 distally in preparation for needle assembly 570 deployment. Once the launch tube has been desirably advanced, locking control 612 may again be partially rotated, as shown in FIG. 21D, such that guides 638 on needle assembly 570 are free to then be advanced within longitudinal needle assembly channel 644 relative to the handle enclosure 610 for deploying the needle assembly 570 from the launch tube and into or through the tissue. As mentioned above, the needle assembly 570 may be removed from enclosure 610 and the locking mechanism by reversing the above procedure.

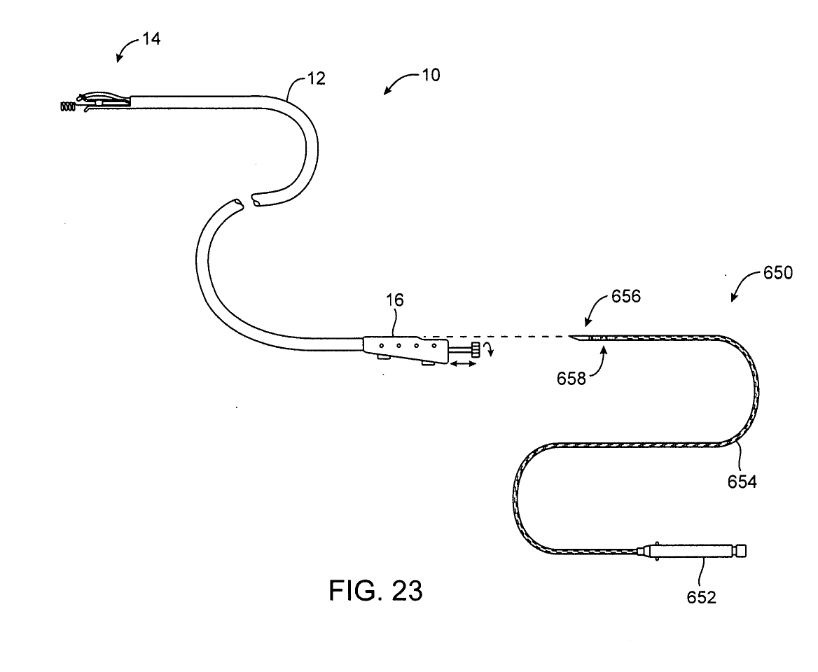

As described above, needle deployment assembly 650 may be deployed through approximation assembly 10 by introducing needle deployment assembly 650 into the handle 16 and through tubular body 12, as shown in the assembly view of FIG. 23, such that the needle assembly 656 is advanced from the launch tube and into or through approximated tissue. Once the needle assembly 656 has been advanced through the tissue, the anchor 30 assembly 658 may be deployed or ejected. Anchor assembly 658 is normally positioned within the distal portion of tubular sheath 654 which extends from needle assembly control or housing 652. Once the anchor assembly 658 has been fully deployed from sheath 654, the spent needle deployment assembly 650 may be removed from approximation assembly 10, as described above, and another needle deployment assembly may be introduced without having to remove assembly 10 from the patient. The length of sheath 654 is such that it may be passed entirely through the length of tubular body 12 to enable the deployment of needle assembly 656 into and/or through the tissue.

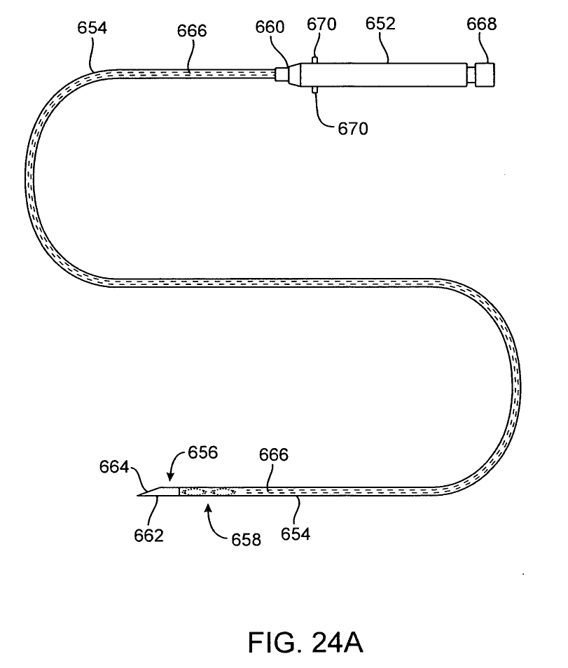

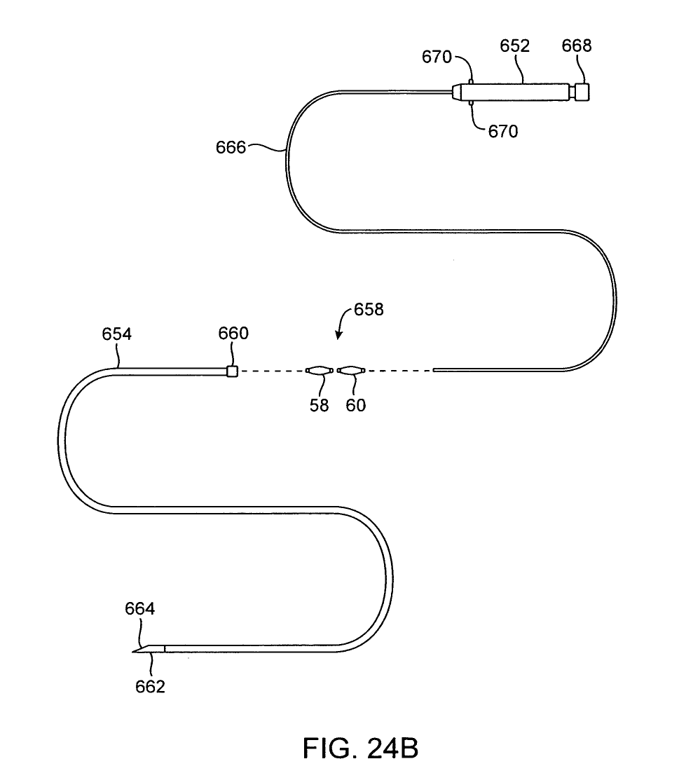

FIG. 24A shows a detailed assembly view of the needle deployment assembly 650 from FIG. 23. In this variation, elongate and flexible sheath or catheter 654 may extend removably from needle assembly control or housing 652. Sheath or catheter 654 and housing 652 may be interconnected via interlock 660 which may be adapted to allow for the securement as well as the rapid release of sheath 654 from housing 652 through any number of fastening methods, e.g., threaded connection, press-fit, releasable pin, etc. Needle body 662, which may be configured into any one of the variations described above, may extend from the distal end of sheath 654 while maintaining communication between the lumen of sheath 654 and needle opening 664.

Elongate pusher 666 may comprise a flexible wire or hypotube which is translationally disposed within sheath 654 and movably connected within housing 652. A proximally-located actuation member 668 may be rotatably or otherwise connected to housing 652 to selectively actuate the translational movement of elongate pusher 666 relative to sheath 654 for deploying the anchors from needle opening 664. Anchor assembly 658 may be seen positioned distally of elongate pusher 666 within sheath 654 for deployment from sheath 654. Needle assembly guides 670 may also be seen protruding from housing 652 for guidance through the locking mechanism described above. FIG. 24B shows an exploded assembly view of the needle deployment assembly 650 from FIG. 24A. As seen, sheath 654 may be disconnected from housing 652 via interlock 660 to reveal the elongate pusher 666 connected to housing 652 and the distal and proximal anchors 58, 60, respectively, of anchor assembly 658.

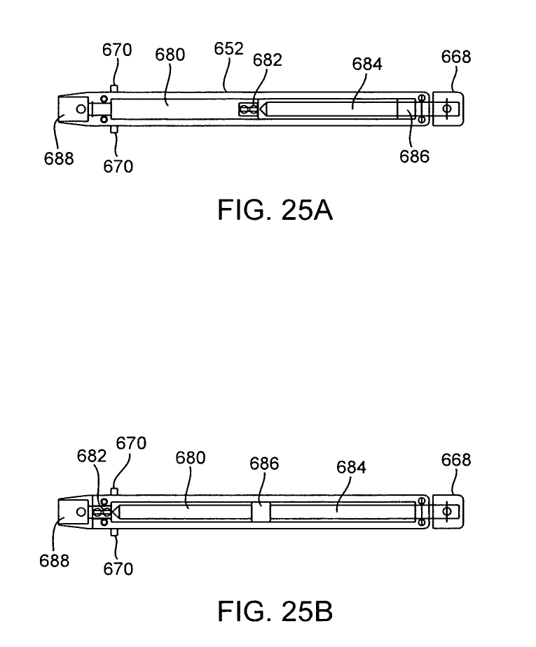

FIGS. 25A and 25B show partial cross-sectional views of one variation of housing 652. As shown in FIG. 25A, elongate pusher 666 may be attached to shuttle 682, which in turn may be connected to threaded interface element 686. As actuation member 668 is manipulated, e.g., by rotating it clockwise, lead screw 684 may be rotated about its longitudinal axis to advance threaded interface element 686 over lead screw 684 distally through shuttle channel 680, as shown in FIG. 25B, where shuttle 682 has been advanced entirely through shuttle channel 680. Tubular sheath interlock 688 may be seen at the distal portion of housing 652 through which the elongate pusher 666 may be advanced. To reverse the direction of elongate pusher 666 and shuttle 682, actuation member 668 may be reversed in the opposite direction.

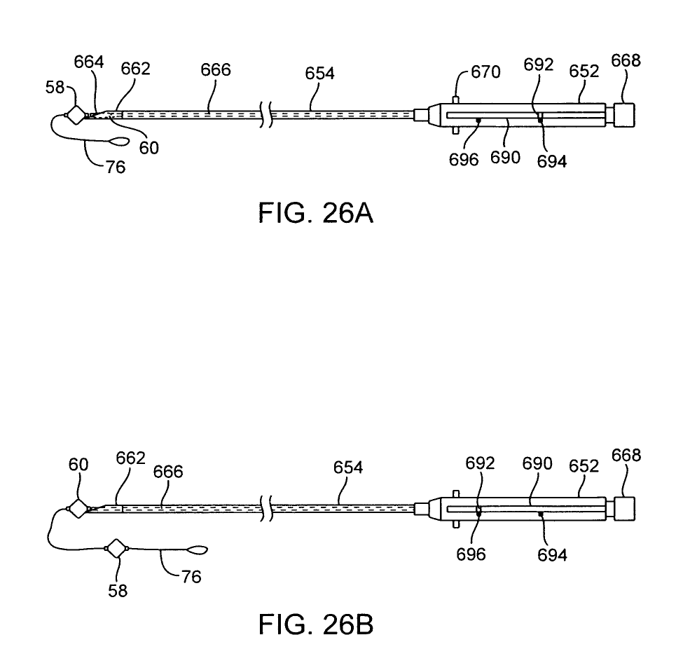

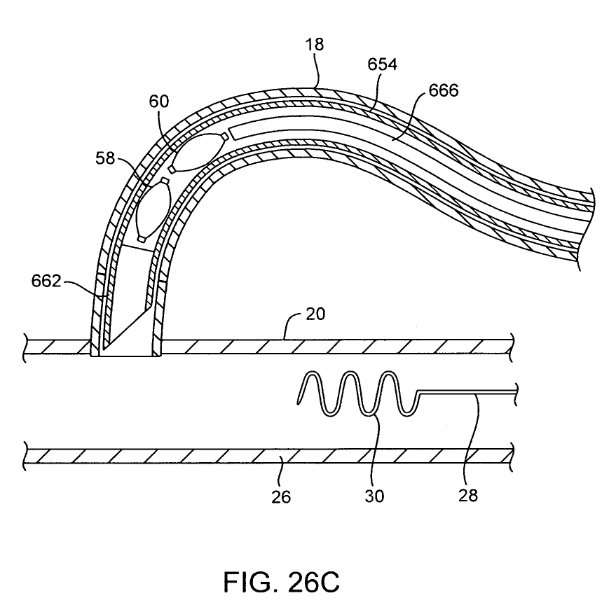

Another variation of the needle deployment assembly may be seen in FIGS. 26A and 26B which show assembly side views. In this variation, housing 652 may define an indicator window 690 along the length of housing 652 to enable viewing of a visual indicator 692 which may be utilized to indicate the position of the elongate pusher 666 within the sheath 654. In the illustration of FIG. 26A, as actuation member 668 is manipulated to advance pusher 666 distally, indicator 692 may move correspondingly within window 690. Positional indicators may also be marked along window 690 to indicate to the user when specified limits have been reached. For instance, positional indicator 694 may be marked such that alignment of indicator 692 with positional indicator 694 is indicative to the user that distal anchor 58 has been deployed from sheath 654.

Likewise, an additional positional indicator 696 may be marked such that alignment of indicator 692 with positional indicator 694 is indicative to the user that the proximal anchor 60 has also been deployed from sheath 654, as shown in FIG. 26B. Any number of positional indicators or methods for visually marking may be utilized as the above examples are merely intended to be illustrative and not limiting. Moreover, to further facilitate the visualization of anchor positioning within sheath 654, the sheath itself may be fabricated from a transparent material, such as plastics, so that the user may visually locate a position of one or both anchors during anchor deployment into or through the tissue.

FIG. 26C shows an illustrative cross-sectional view of the launch tube 18 in its deployment configuration. Tubular sheath 654 and needle body 662 may be seen positioned within the distal portion of launch tube 18 ready for deployment into any tissue (not shown for clarity) which may be positioned between upper and lower extension members 20, 26. Also shown are distal and proximal anchors 58, 60, respectively (suture is not shown for clarity), positioned within sheath 654 distally of elongate pusher 666.

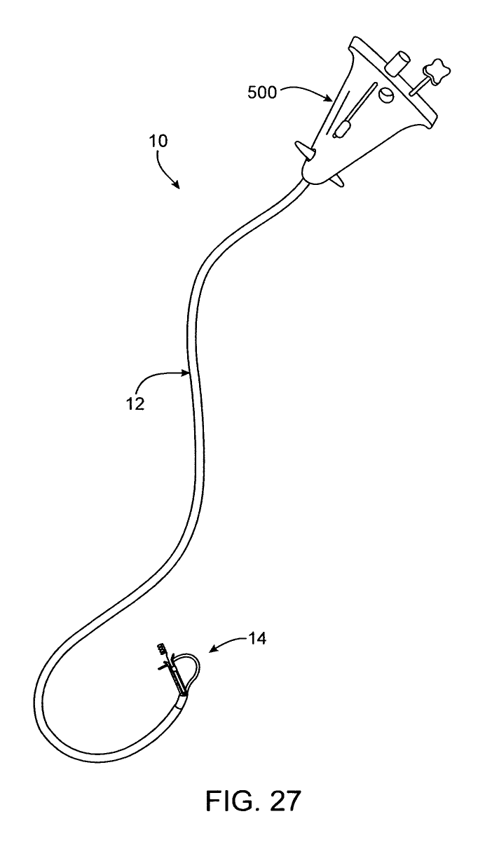

Various force transmission elements or configurations may be provided to actuate elements of end effectors. Such end effectors may, for example, comprise previous described end effector 14, or any alternative medical end effector. Referring to FIG. 27, an embodiment of apparatus 10 is provided comprising flexible tubular body 12 that couples end effector 14 to handle 500. Force transmission elements, such as those described previously and/or those described hereinafter, optionally may be integrated into, and/or actuable via, the handle.

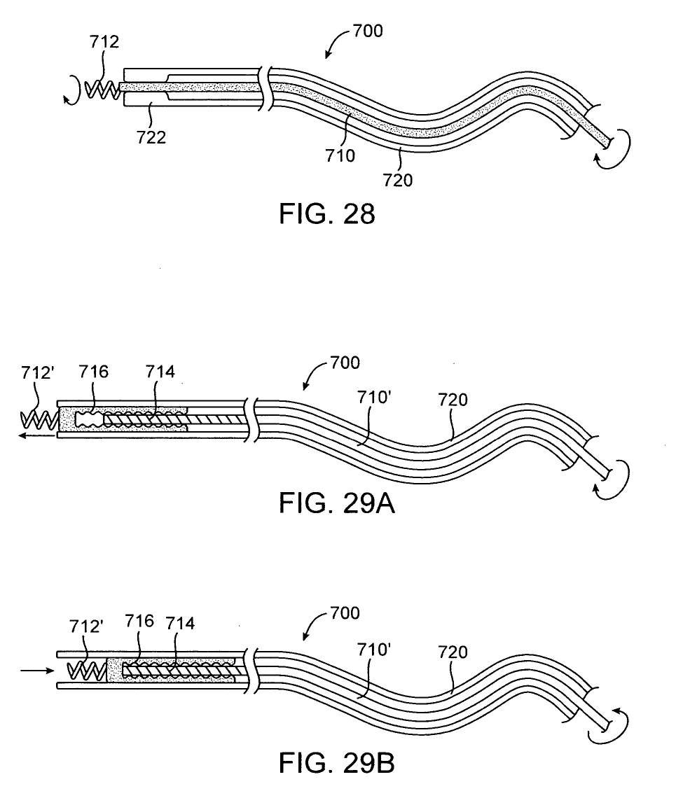

With reference to FIG. 28, a first embodiment of such a force transmission element illustratively is shown actuating a tissue acquisition member that may, for example, be utilized as part of end effector 14 of apparatus 10. As will be apparent, the force transmission element (as with other force transmission elements described hereinafter) optionally may be utilized to actuate other elements of end effector 14 of apparatus 10, or of some other medical end effector. Tissue acquisition member 700 comprises elongated member 710 disposed within outer sheath 720. Outer sheath 720 optionally may comprise locally necked-down distal region 722 that acts as a bearing surface for rotation and/or translation of elongated member 710. Elongated member 710 comprises distal tissue grasper 712, illustratively a helical tissue grasper. As illustrated by arrows in FIG. 28, rotation of a proximal region of member 710 transmits a rotational torque to distal tissue grasper 712. Likewise, translation of the proximal region translates the grasper. Member 710 optionally may be translationally (or rotationally) fixed relative to outer sheath 720, e.g., fixed at necked down distal region 722 of the outer sheath. It should be understood that outer sheath 720 optionally may comprise the working channel of an endoscope or other medical instrument, per se known.

Referring now to FIG. 29, a force transmission element that transmits and converts rotational motion into translation motion via a lead screw mechanism is described. In FIG. 29, tissue acquisition member 700 comprises elongated member 710′ having distal lead screw 714. Tissue grasper 712′ comprises mating screw 716. As seen in FIG. 29A, rotation of a proximal region of member 710′ in a first direction translationally advances tissue grasper 712′ relative to outer sheath 720 via the lead screw coaction of distal screw 714 of elongated member 710′ with mating screw 716 of tissue grasper 712′. Likewise, as seen in FIG. 29B, rotation of the proximal region of member 710′ in the opposite direction actuates the lead screw to translationally retract grasper 712′ relative to outer sheath 720.

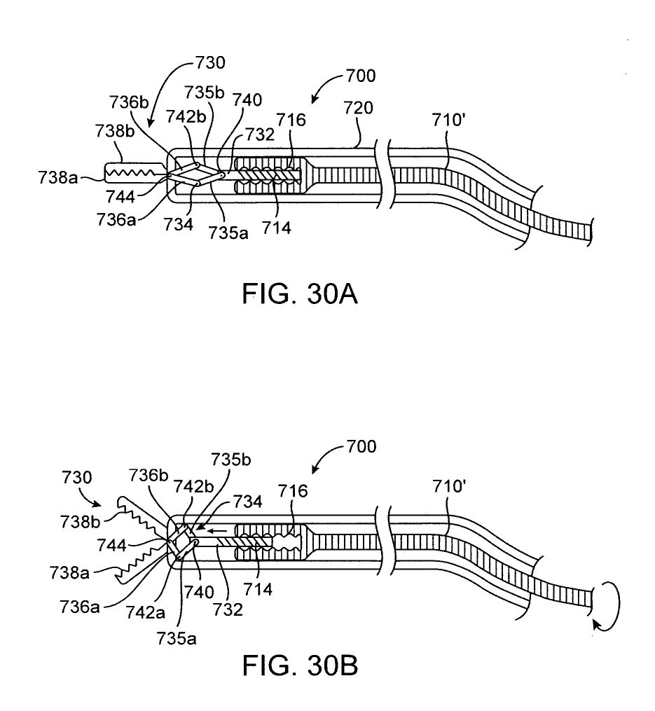

With reference to FIG. 30, tissue acquisition member 700 converts rotational motion into translational motion that actuates a linkage to initiate a more complex motion. In FIG. 30, the male and female elements of the lead screw have been reversed. Specifically, tissue grasper 730 comprises member 732 having male screw 714, while elongated member 710′ comprises female mating screw 716. It should be understood that the screw elements may be reversed, as desired.

Tissue grasper 730 may further comprise four bar linkage 734 having first and second bars 735a and 735b, respectively, that are coupled at pivot 740 to member 732. The four bar linkage further comprises third and fourth bars 736a and 736b, respectively, that are coupled to the first and second bars at pivots 742a and 742b, respectively. The third and fourth bars cross and are pivotally attached to one another, as well as to sheath 720, at pivot 744. First and second jaw members 738a and 738b extend from (or are integrally formed with) the third and fourth bars, respectively, for grasping tissue.

As seen in FIG. 30A, rotation of a proximal region of member 710′ in a first direction translationally advances member 732 of tissue grasper 730 relative to sheath 720 and/or elongated member 710′ via the coacting lead screw. Advancement of member 732 actuates four bar linkage 734 in a manner that separates and opens jaw members 738a and 738b, e.g., for engaging or releasing engaged tissue. As seen in FIG. 30B, rotation of member 710′ in an opposite direction translationally retracts member 732 of grasper 730 relative to sheath 720/member 710′. This actuates four bar linkage 734 in a manner that approximates and closes jaw members 738, e.g., to secure engaged tissue therebetween or to provide a lower profile delivery or retrieval configuration.

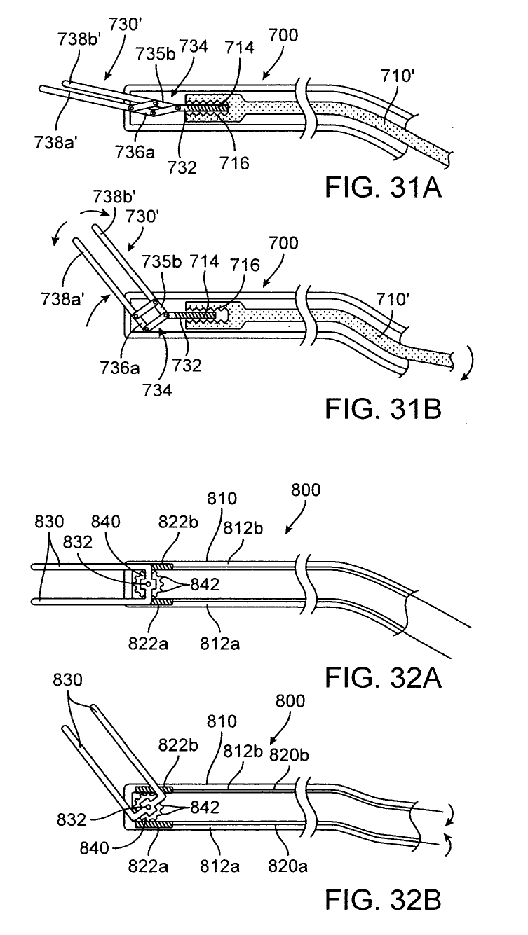

Referring to FIG. 31, an alternative embodiment of the apparatus of FIG. 30 is described. In FIG. 31, tissue acquisition member 700 comprises tissue manipulation assembly 730′ rather than tissue grasper 730. Specifically, jaw members 738 of grasper 730 have been replaced with first and second extension members 738′. First extension member 738a′ may extend from third bar 736a of four bar linkage 734, while second extension member 738b′ may likewise extend from second bar 735b of the linkage. As seen in FIG. 31, rotation of member 710′ advances or retracts member 732, which actuates four bar linkage 734 and reorients the extension members relative to sheath 720.

In the embodiment of FIG. 31, a separation distance between the extension members may vary during actuation of linkage 734 and reorientation of the extension members. FIG. 32 provide apparatus and a method for coordinated reorientation or pivoting of extension members of a tissue manipulation assembly, whereby the separation distance between the extension members does not vary. Apparatus 800 comprises sheath 810 having first and second guide lumens 812a and 812b, respectively, disposed within the wall of the sheath. Elongated members 820a and 820b having first and second lead screws 822a and 822b, respectively, are disposed within guide lumens 812. Extension members 830 are integrally formed into a U-shaped structure that is connected to gear 840 at attachment 832. Attachment 832 may pivotably attach the gear and extension members to sheath 810. Gear 840 comprises teeth 842 that are configured to coact with lead screws 822.

As illustrated by arrows in FIG. 32B, coordinated rotation of elongated members 820a and 820b in opposing directions pivots or reorients extension members 830 relative to sheath 810 via coaction of gear teeth 842 with lead screws 822. As will be apparent, extension members 830 alternatively may be reoriented via coaction of gear 840 with a single lead screw 822. Furthermore, a medical practitioner may actively rotate only a single elongated member 820, and the secondary elongated member 820 may passively rotate in an opposing direction via interaction of its lead screw with the gear. Furtherstill, the first and second elongated members 820 may be rotated in the same direction, or one of the elongated members may be held stationary while the other is rotated, in order to friction lock an orientation or position of extension members 830 relative to sheath 810.

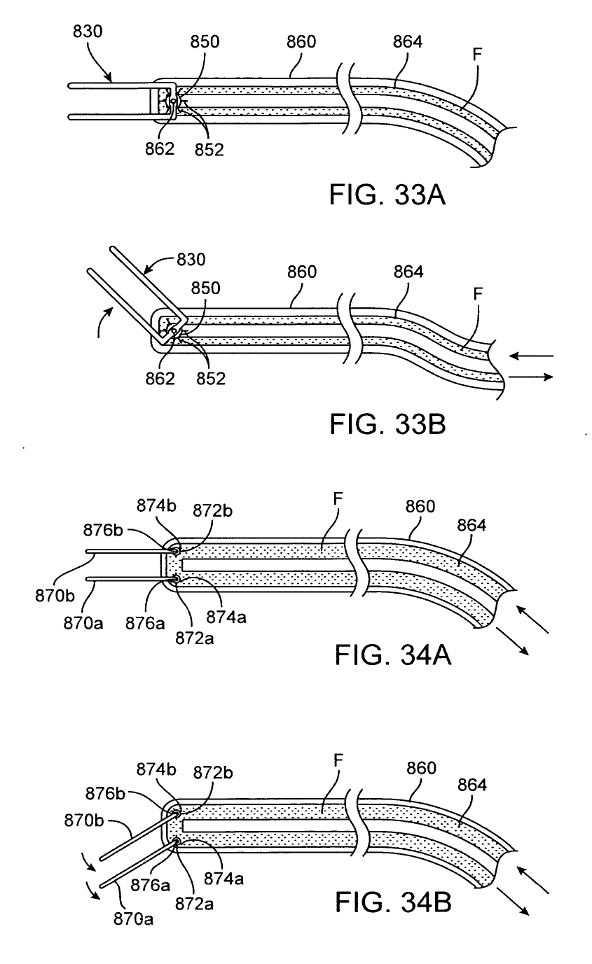

Referring now to FIG. 33, hydraulic rotation of extension members 830 is described. In FIG. 33, extension members 830 are coupled to fluid wheel or turbine 850. Fluid wheel 850 comprises multiple extensions or spokes 852 that facilitate hydraulic rotation of the wheel. The fluid wheel and extension members 830 may be pivotably attached to sheath 860 at pivot 862. Sheath 860 comprises fluid channel 864 having fluid F disposed therein. Spokes 852 of fluid wheel 850 communicate with channel 864. As illustrated by arrows in FIG. 33B, fluid F may be forced through channel 864 under pressure to apply a hydraulic moment to spokes 852 of wheel 850 that rotates the wheel about pivot 862 in the direction of fluid flow. Rotation of wheel 850 rotates and reorients extension members 830 that are attached to the wheel relative to sheath 860.

With reference to FIG. 34, independent hydraulic rotation of each of the extension members is described. Extension members 870a and 870b comprise fluid wheels 872a and 872b, respectively, having spokes 874a and 874b, respectively. Wheels 872a and 872b are pivotably coupled to sheath 860 at pivots 876a and 876b, respectively, which are disposed in fluid channel 864 of sheath 860. Pressurized flow of fluid F through channel 864 applies hydraulic moments to spokes 874a and 874b of the fluid wheels that rotate the wheels about pivots 876 in the direction of fluid flow. Rotation of wheels 872a and 872b independently rotates and reorients extension members 870a and 870b relative to sheath 860.

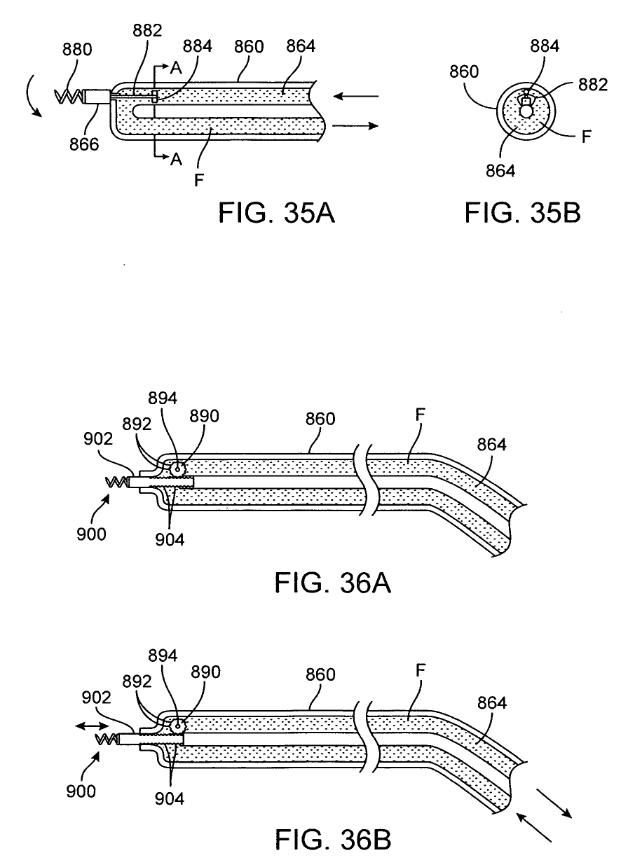

Referring now to FIG. 35, hydraulic actuation of a tissue acquisition member or tissue grasper is described. Helical tissue grasper 880 comprises shaft 882 having propeller 884 disposed within fluid channel 864 of sheath 860. Helical grasper 880 is configured for rotation within extension 866 of sheath 860. Pressurized flow of fluid F through channel 864 rotates propeller 884, which in turns rotates helical tissue grasper 880. Fluid F may, for example, flow through channel 864 in a first direction to rotate helical grasper 880 in a direction appropriate for engaging tissue, and may flow in an opposing direction to rotate the helical grasper in an opposing direction appropriate for disengaging the tissue.

With reference to FIG. 36, fluid wheel or gear 890 having spokes or teeth 892 is pivotably coupled to sheath 860 at pivot 894 disposed within channel 864. Helical grasper 900 comprises shaft 902 having proximal corrugations or protrusions 904 that are configured to coact with teeth 892 of fluid gear 890. As illustrated in FIG. 36B, pressurized flow of fluid F in a first direction through channel 864 applies a moment to teeth 892 of gear 890 that rotates the gear about pivot 894. This rotation advances helical grasper 900 relative to sheath 860 via coaction of teeth 892 of gear 890 with corrugations 904 of shaft 902 of grasper 900. Fluid flow through channel 864 in an opposing direction would retract grasper 900 relative to sheath 860 in a similar fashion.

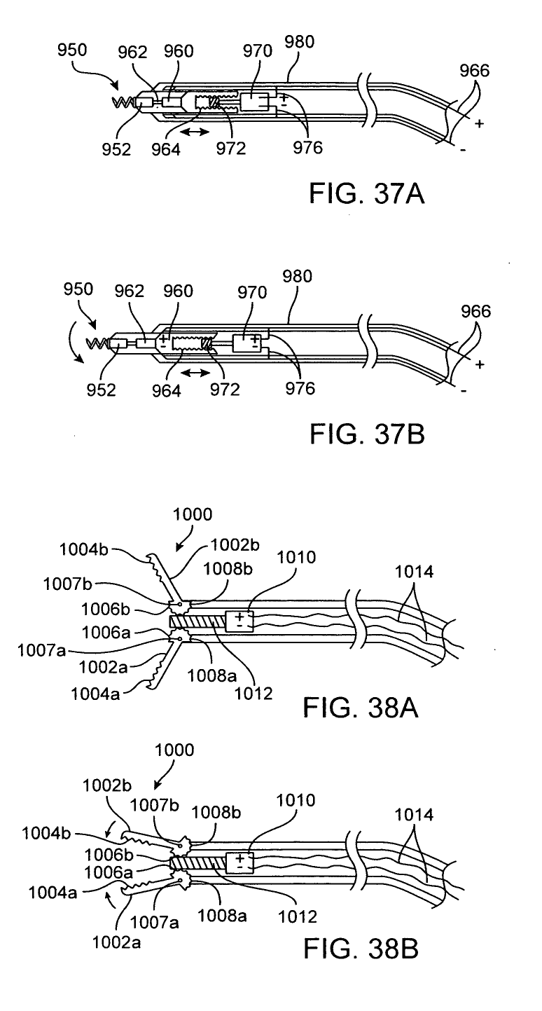

Referring now to FIG. 37, motor-actuated force transmission elements for advancing and rotating an end effector element are described. Helical tissue acquisition member or grasper 950 comprises shaft 952 that is proximally coupled to drive shaft 962 of first electric motor 960. Motor 960 is slidably disposed within sheath 980 and comprises mating screw 964 that is configured to coact with lead screw drive shaft 972 of second electric motor 970. Second motor 970 is coupled to sheath 980. First motor 960 comprises positive and negative electrical hook-ups 966, while second motor 970 comprises electrical hook-ups 976.

A current passed through first motor 960 via electrical hook-ups 966 rotates the motor's drive shaft 962, which rotates helical grasper 950. Reversing the polarity of current passed through motor 960 reverses the direction of rotation of grasper 950. Passage of a current through second motor 970 via electrical hook-ups 976 rotates lead screw drive shaft 972, which coacts with mating screw 964 of first motor 960 to advance or retract the first motor relative to sheath 980, thereby advancing or retracting helical tissue grasper 950 relative to the sheath.

With reference to FIG. 38, a motor-actuated jaw tissue grasper is described. Tissue grasper 1000 comprises first and second jaws 1002a and 1002b, respectively, having interdigitating distal teeth 1004 for engaging tissue. Jaws 1002 further comprise proximal gears 1006 having teeth 1008 that are configured to coact with lead screw drive shaft 1012 of electric motor 1010. Gears 1006 are pivotably connected to sheath 1016 at pivots 1007. Motor 1010, which is coupled to sheath 1016, comprises electrical hook-ups 1014, and passage of an electrical current through the motor via the hookups rotates lead screw drive shaft 1012. Coaction of gear teeth 1008 with the rotating lead screw acts to approximate or separate first and second jaws 1002, depending on the polarity of the current passed through the motor.

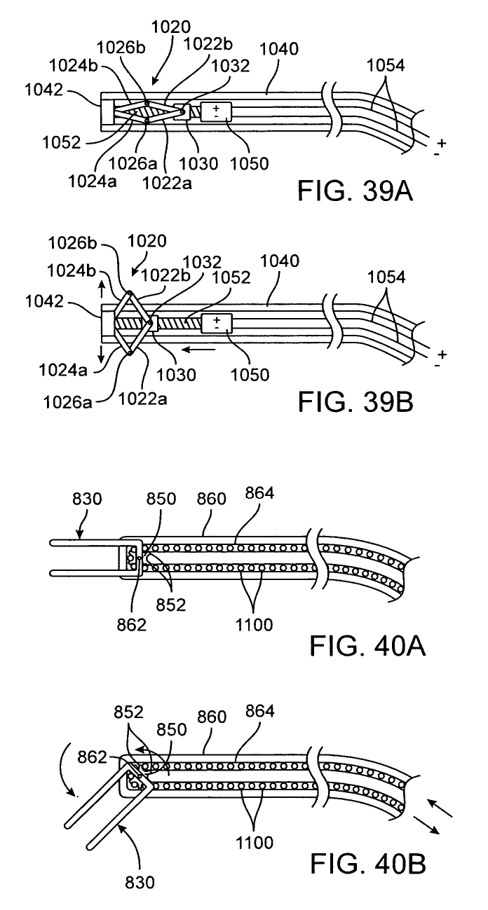

Referring to FIG. 39, a motor-actuated four-bar linkage is described. Linkage 1020 comprises first and second bars 1022a and 1022b, respectively, that are coupled at pivot 1032 to nut member 1030. The four bar linkage further comprises third and fourth bars 1024a and 1024b, respectively, that are coupled to the first and second bars at pivots 1026a and 1026b, respectively. The third and fourth bars cross and are pivotably attached to one another, as well as to sheath 1040, at pivot 1042. Sheath 1040 comprises through-holes, side-ports or windows (not shown) that accommodate expansion of four bar linkage 1020.

Nut member 1030 is concentrically disposed about, and comprises a mating screw adapted to coact with, lead screw drive shaft 1052 of electric motor 1050. Motor 1050 is coupled to sheath 1040, and it comprises electrical hook-ups 1054. Passage of an electrical current through the motor via the hook-ups rotates lead screw drive shaft 1052, which advances or retracts nut member 1030 relative to the drive shaft, dependent on the direction of rotation of the drive shaft. As seen in FIG. 39B, advancement of the nut member actuates linkage 1020 in a manner that shortens and expands the linkage.

Referring now to FIG. 40, a force transmission element comprising a column of ball-bearings is described. The apparatus of FIG. 40 is substantially the same as the apparatus of FIG. 33, except that channel 864 of sheath 860 is filled with collinearly-aligned ball-bearings 1100, rather than fluid F. As illustrated by arrows in FIG. 40B, the column of ball-bearings 1100 may be pushed through channel 864 to apply a moment to spokes 852 of wheel 850 that rotates the wheel about pivot 862 in the direction of motion of the ball-bearing column. Rotation of wheel 850 rotates and reorients extension members 830 that are attached to the wheel relative to sheath 860.

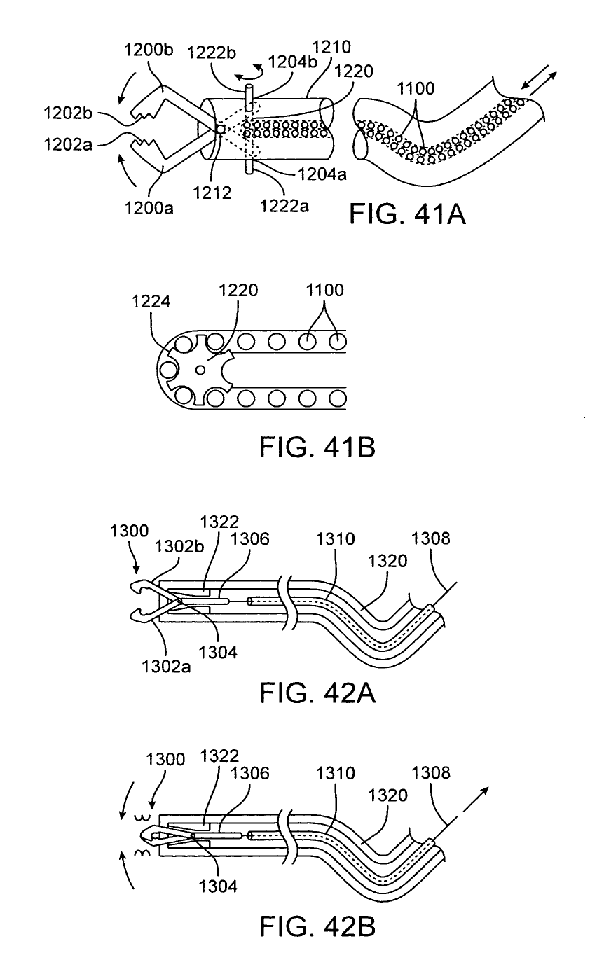

With reference now to FIG. 41, crimping or grasping via a ball-bearing column is described. Crimping jaws 1200a and 1200b are pivotably connected to one another and to sheath 1210 at pivot 1212. Each crimping jaw comprises a distal crimping surface 1202 and a proximal mating screw 1204. The proximal mating screws are coaxially disposed over rod 1220 having first and second oppositely-turned lead screws 1222a and 1222b that are configured to coact with mating screws 1204. Rod 1220 is rotatably coupled to sheath 1210, and rotation of the rod causes crimping jaws 1200a and 1200b to move in opposite directions (either towards one another or away from one another) via the lead screws. The previously described column of ball-bearings 1100 is also provided, either with a channel of sheath 1210 or within their own malleable sleeve. The column of ball-bearings extends around and contacts a central region of rod 1220.

As seen in the detail view of FIG. 41, the central region of rod 1220 comprises profiled surface 1224 having multiple divots configured for placement of a ball bearing therein. In this manner, ball-bearing column 1100 engagingly contacts rod 1220, such that movement of the column rotates the rod. As mentioned, such rotation opens or closes jaws 1200, dependent upon the direction of rotation. Jaws 1200 may, for example, be spread apart for placement of a crimp therebetween, then approximated to deform the crimp. Such crimping may be controlled from a proximal location by a medical practitioner via the column of ball-bearings.

Referring now to FIG. 42, a force transmission mechanism utilizing geometric constraints is described. Grasper or crimper 1300 comprises jaws 1302a and 1302b that are pivotably joined at pivot 1304 and are biased into a spread or open configuration, e.g. via a spring. Proximal extension 1306 extends from pivot 1304, and wire 1308 extends proximally from extension 1306. Wire 1308 extends through tube 1310. Grasper 1300 is disposed within sheath 1320 having conical or wedge-shaped distal insert 1322 through which proximal extension 1306 of the grasper extends.

Jaws 1302 of grasper 1300 may be advanced out of sheath 1320 by advancing tube 1310 against extension 1306 of the grasper. Such advancement of the grasper may be achieved by a medical practitioner advancing a proximal portion of the tube disposed outside of a patient. As seen in FIG. 42A, jaws 1302 spread apart to their biased, open configuration. The jaws then may be approximated, e.g., to engage tissue or deform a crimp, by retracting wire 1308 from outside the patient, such that the jaws contact distal insert 1322 of sheath 1320 and are urged together into an approximated configuration, as in FIG. 42B.

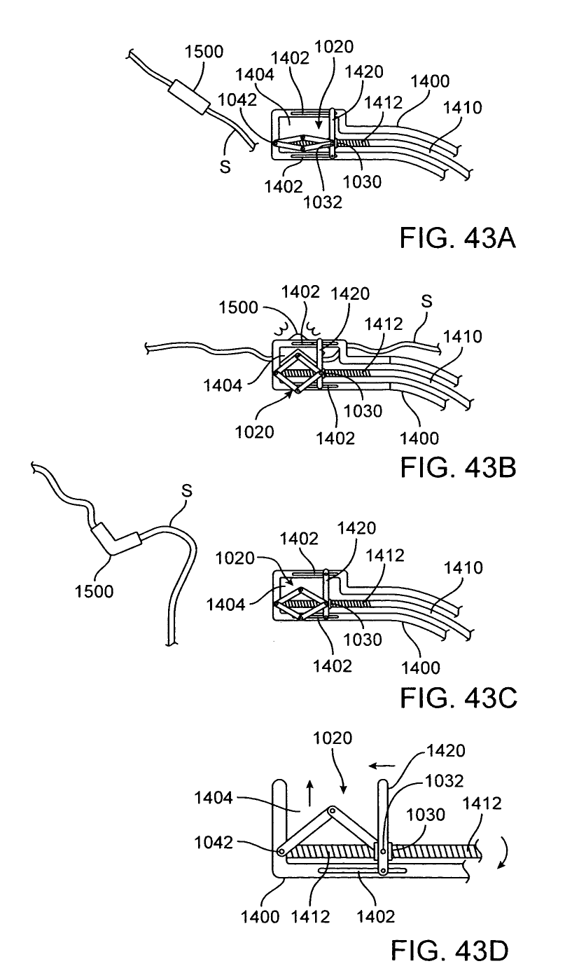

Referring now to FIG. 43, a method of deforming a crimp with a linkage assembly is described. The apparatus of FIG. 43 is similar to that of FIG. 39. Previously-described linkage 1020 is proximally coupled at pivot 1032 to nut member 1030, and is distally coupled at pivot 1042 to sheath 1400. Nut member 1030 is concentrically disposed about, and comprises a mating screw adapted to coact with, lead screw 1412 of elongated member 1410. Extension member 1420 is coupled to nut member 1030 and is slidably disposed within linear bearings 1402 of sheath 1400. Rotation of elongated member 1410 advances or retracts nut member 1030 along the lead screw, which, in turn, advances or retracts extension member 1420 and expands or collapses linkage 1020.

As seen in FIG. 43A, a distal end of sheath 1400 may be disposed in proximity to crimp 1500 having suture S running therethough. In FIG. 43B, the crimp may be disposed within open chamber 1404 of the sheath and may be deformed by rotating elongated member 1410 to actuate the lead screw, which expands linkage 1020 and urges member 1420 against the crimp. Linkage 1020 then may be collapsed, and member 1420 may be moved proximally, by rotating elongated member 1410 in the opposite direction to actuate the lead screw in a manner that retracts nut member 1030 relative to sheath 1400. As seen in FIG. 43C, deformed crimp 1500 then may be removed from chamber 1404. Thereafter, the deformed crimp will maintain the position of suture S relative to the crimp. As seen in the detail view of FIG. 43D, a similar deformation mechanism may be achieved with a two bar embodiment of linkage 1020, as well as with the top portion of chamber 1404 and/or at least one of the linear bearings 1402 removed.

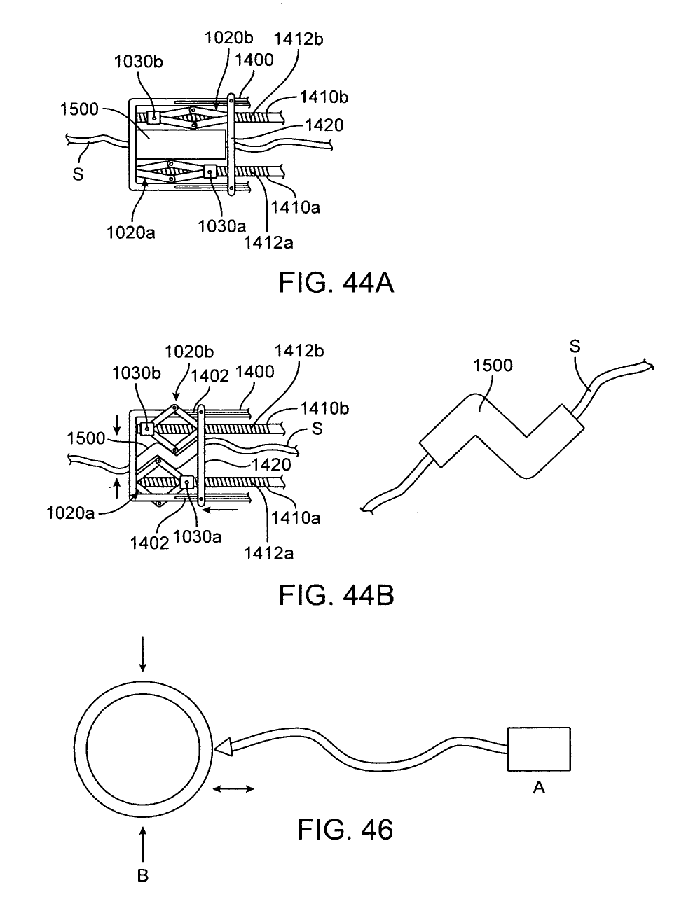

With reference to FIG. 44, an alternative embodiment of the apparatus and method of FIG. 43 is described. As seen in FIGS. 43B and 43C, linkage 1020 may be used to form a single kink in crimp 1500. However, multiple linkages may be provided to form multiple kinks in the crimp. It is expected that providing multiple kinks in the crimp will produce a more tortuous path through the crimp, e.g., a more tortuous path for passage of suture S through crimp 1500 that will better maintain the position of the suture relative to the crimp.

In FIG. 44, first and second linkages 1020a and 1020b illustratively are provided to form first and second kinks or bends in crimp 1500. First and second elongated members 1410 having first and second lead screws 1412 are also provided. As illustrated in FIG. 44, the linkages may be coupled to extension member 1420, or may move independently along the lead screws via nut members 1030. As seen in FIG. 44A, crimp 1500 may be disposed between linkages 1020a and 1020b. The linkages then may be expanded to deform the crimp with multiple kinks or bends, as in FIG. 44B.

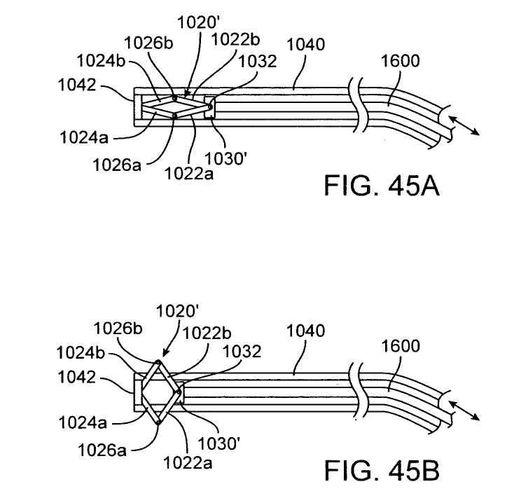

Referring now to FIG. 45, a four-bar linkage actuated via linear or translational motion is described. Linkage 1020′ is similar to linkage 1020 and comprises first and second bars 1022a and 1022b, respectively, that are coupled at pivot 1032 to piston member 1030′. The four bar linkage further comprises third and fourth bars 1024a and 1024b, respectively, that are coupled to the first and second bars at pivots 1026a and 1026b, respectively. The third and fourth bars cross and are pivotably attached to one another, as well as to sheath 1040, at pivot 1042. Sheath 1040 comprises through-holes, side-ports or windows (not shown) that accommodate expansion of four bar linkage 1020′.

Piston member 1030′ is coupled to push-pull member 1600, which extends through sheath 1040 to a proximal region, where it may be manipulated by a medical practitioner. As seen in FIG. 45B, advancement of push-pull member 1600 relative to sheath 1040 advances piston member 1030′, which in turn actuates linkage 1020′ in a manner that shortens and expands the linkage. Subsequent retraction of member 1600 relative to the sheath retracts the piston member, which elongates and collapses the linkage back to the delivery configuration of FIG. 45A. As will be apparent, jaw members or graspers, extension members, or any other end effector may be coupled to, and/or actuated by, linkage 1020′.

A variety of transmission mechanisms have been described for transmitting force, energy and/or power along desired vectors over significant distances from a medical practitioner to an end effector. It should be understood that the embodiments are provided for illustration only, and elements of the embodiments may be used in any combination as practicable. FIG. 46 provides a schematic representation for a generic transmission mechanism. A medical practitioner positioned at location A transmits force, energy and/or power to an end effector disposed at position B. The direction or type of the force/power/energy may be converted at or in the vicinity of position B to a form or direction appropriate for actuating the end effector. For example, force may be converted from rotational to translational, or vice versa. Additionally or alternatively, energy may be converted from electrical or fluid to mechanical, etc.

A variety of mechanisms, per se known, may be utilized to transmit force/power/energy from the medical practitioner to the end effector. These include, but are not limited to, hydraulic pumps; fluid compressors; pressure tanks; condensate separators and drain valves; compressed air systems, regulators or valves; hydraulic cylinders; electromechanical and/or linear actuators and solenoids; electric or air motors; speed reducers; roller chains; sprockets and bushings; clutches and torque limiters; timing and drive belts or pulleys; linear, rotational, plain, ball, tapered, needle, thrust or mounted bearings; lead screws; ball screws; linear motion; track or drive rollers; screw jacks; turntables; shaft collars or couplings; universal joints; rod ends and linkages; devises; control cables; gas springs; shock absorbers; encoders; pistons; etc. Additional known mechanisms will be apparent to those of skill in the art.

Although a number of illustrative variations are described above, it will be apparent to those skilled in the art that various changes and modifications may be made thereto without departing from the scope of the invention. Moreover, although specific configurations and applications may be shown, it is intended that the various features may be utilized in various types of procedures in various combinations as practicable. It is intended in the appended claims to cover all such changes and modifications that fall within the true spirit and scope of the invention.

What is claimed is:

1. Apparatus for conveying force or energy to a medical end effector, the apparatus comprising:
an elongate member having a proximal end and a distal end, and a length therebetween, the medical end effector positioned at the distal end of the elongate member; and
a transmission mechanism configured to transfer force or energy between the proximal end of the elongate member and the medical end effector,
wherein the transmission mechanism is configured to alter the direction or form of the force or energy as it is transferred between the proximal end of the elongate member and the medical end effector, and
wherein the medical end effector is configured to fold tissue.

2. The apparatus of claim 1, wherein the transmission mechanism is configured to alter translational force or energy into rotational force or energy.

3. The apparatus of claim 1, wherein the transmission mechanism configured to alter rotational force or energy into translational force or energy.

4. The apparatus of claim 1, wherein the transmission mechanism configured to alter hydraulic force or energy into mechanical force or energy.

5. The apparatus of claim 1, wherein the transmission mechanism comprises a lead screw.

6. The apparatus of claim 1, wherein the transmission mechanism comprises a column of ball-bearings.

7. The apparatus of claim 1, wherein the medical end effector comprises a tissue acquisition member.

8. The apparatus of claim 1, wherein the medical end effector comprises a tissue engagement member.

9. The apparatus of claim 1, wherein the medical end effector comprises a tissue grasper.

10. The apparatus of claim 1, wherein the medical end effector comprises a tissue securement element for securing folded tissue.

11. The apparatus of claim 1, wherein the medical end effector comprises a crimping element.

12. The apparatus of claim 1, wherein the medical end effector comprises a tissue manipulation assembly having extension members for folding tissue therebetween.

13. The apparatus of claim 1, wherein the medical end effector comprises a linkage.

14. The apparatus of claim 1, wherein the medical end effector comprises a gear.

15. The apparatus of claim 1, wherein the medical end effector comprises a turbine.

16. The apparatus of claim 1, wherein the elongate member is flexible, and wherein the apparatus is configured for endoluminal placement of the end effector within a patient.

17. The apparatus of claim 1, wherein the apparatus is configured for laparoscopic placement of the end effector within a patient.

18. A method for performing a medical procedure with a medical end effector disposed at a distal end of an elongate member, the method comprising:
advancing the medical end effector into a patient;
transmitting force or energy to the medical end effector from a proximal region of the elongate member disposed outside the patient;
altering the force or energy as it is transmitted to the medical end effector, and
performing the medical procedure with the end effector via the altered force or energy,
wherein performing the medical procedure comprises folding tissue.

19. The method of claim 18, wherein advancing the medical end effector into a patient further comprises endoluminally advancing the end effector into the patient.

20. The method of claim 18, wherein advancing the medical end effector into a patient further comprises laparoscopically advancing the end effector into the patient.

21. The method of claim 18, wherein altering the force or energy further comprises altering the direction or form of the force or energy.

22. The method of claim 18, wherein altering the force or energy comprises the altering the force or energy from translational to rotational force or energy.

23. The method of claim 18, wherein altering the force or energy comprises the altering the force or energy from rotational to translational force or energy.

24. The method of claim 18, wherein altering the force or energy comprises the altering the force or energy from hydraulic to mechanical force or energy.

25. The method of claim 18 wherein performing the medical procedure further comprises securing the folded tissue.
